SEQUENCE LISTING (9 sequences)

SEQ ID NO: 1 - DNA, 385 bp, Artificial
  Synthetic nucleotide
gtaaagatgctcaagcgtaagttcctgtatggtactgcatgcgcttgacatcagtttgccagttgtgctttttgctaaaatgcatgtttccaattttagcgagtgcccatttgacagaacgggaagccctcatgtctgaactcaaagtcctgagttaccttggtaatcacatgaatattgcgaatctacttggagcctgcaccattggaggtaaagccgtgtccaagctgccttttattgtctgtcaggttatcaaaacatgacattttaatatgattttggcaatgctagattataaactgcttggaagatttttttacccagactgttgttctctcttgctagattttgttttcctcattgttcttaagaatgcagattttaa

SEQ ID NO: 2 - DNA, 384 bp, Artificial
  Synthetic nucleotide
taaagatgctcaagcgtaagttcctgtatggtactgcatgcgcttgacatcagtttgccagttgtgctttttgctaaaatgcatgtttccaattttagcgagtgcccatttgacagaacgggaagccctcatgtctgaactcaaagtcctgagttaccttggtaatcacatgaatattgtgactctacttggagcctgcaccattggaggtaaagccgtgtccaagctgccttttattgtctgtcaggttatcaaaacatgacattttaatatgattttggcaatgctagattataaactgcttggaagatttttttacccagactgttgttctctcttgctagattttgttttcctcattgttcttaagaatgcagattttaa

SEQ ID NO: 3 - DNA, 385 bp, Artificial
  Synthetic nucleotide
ttttatgttactccacataaggctgcttttttgataagcagtgttaatatatgggattgtattgggactaagtagtctgatccactgaagctgaatattaatggccatgaccacccttgggtatttttatgggaggcagaattaatctatatatctcaccttctttctaaccttttcttatgtgcttttagggcccaccctggtcattatagaatattgttgctatggtgatcttttgaattttttgagaagaaaacgtgattcatttatttgttcaaagcaggaagatcatgcagaagctgcactttataagaatcttctgcattcaaaggagtcttcctggtaagactgatttacataaatagttagctgttgacaggcagtt

SEQ ID NO: 4 - DNA, 5190 bp, Homo sapiens
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgcgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 5 - DNA, 5190 bp, Homo sapiens
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgactctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 6 - DNA, 5190 bp, Homo sapiens
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattatagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 7 - protein, 976 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIANLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 8 - protein, 976 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVTLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 9 - protein, 976 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVIIEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

(19) United States
(12) Patent Application Publication
(10) Pub. No.: US 2022/0010382 A1
(43) Pub. Date: Jan. 13, 2022

(54) AVAPRITINIB RESISTANCE OF KIT MUTANTS

(71) Applicant: Blueprint Medicines Corporation, Cambridge, MA (US)

(72) Inventor: Oleg Schmidt-Kittler, Cambridge, MA (US)

(21) Appl. No.: 17/292,965

(22) PCT Filed: Nov. 11, 2019
(86) PCT No.: PCT/US2019/060769

§ 371 Date: May 11, 2021

Related U.S. Application Data
(60) Provisional application No. 62/758,806, filed on Nov. 12, 2018.

(51) Int. Cl. C12Q 1/6886; A61K 31/496; A61P 35/02

(52) U.S. Cl. CPC C12Q 1/6886; A61K 31/496; C12Q 2600/156; C12Q 2600/106; A61P 35/02

(57) ABSTRACT

The disclosure includes methods of treating a patient suffering from a malignant disease driven by activating mutations in KIT, said method comprising: (a) obtaining a biological sample from the patient; (b) detecting the presence or absence of a KIT mutation selected from V654A in exon 13, N655T in exon 13, and T670I in exon 14 in the biological sample; and (c) administering a KIT inhibitor to the patient if the mutation is not detected.

### CROSS REFERENCE TO RELATED APPLICATION

This application claims the benefit of priority to U.S. Provisional Application No. 62/758,806, filed Nov. 12, 2018, the entire contents of which are incorporated herein by reference.

### BACKGROUND

The disclosure relates in part to methods for treating a patient suffering from a malignant disease driven by activating mutations in KIT, e.g. a patient suffering from a cancer such as gastrointestinal stromal tumor (GIST). GISTs are the most common malignant subepithelial lesions of the gastrointestinal tract, and the most common symptoms of GISTs are gastrointestinal bleeding, acute melena (dark feces containing blood), hematemesis (vomiting of blood) with anemia, weakness, and abdominal pain and distension. See, Akahoshi et al., Current Clinical Management of Gastrointestinal Stromal Tumor (2018).

The KIT receptor belongs to the class III receptor tyrosine kinase (RTK) family that also includes the structurally related proteins PDGFRA (platelet-derived growth factor receptor A), PDGFRB (platelet-derived growth factor receptor B), FLT3 (FMS-like tyrosine kinase 3), and CSF1R (colony-stimulating factor 1 receptor). Normally, stem cell factor (SCF) binds to and activates KIT by inducing dimerization and autophosphorylation, which induces initiation of downstream signaling. In several tumor types, however, somatic activating mutations in KIT drive ligand-independent constitutive activity; these mutations have been most extensively studied in GIST. Nearly 80% of metastatic GISTs have a primary activating mutation in either the extracellular region (exon 9) or the juxtamembrane (JM) domain (exon 11) of KIT. The recognition that many mutant KIT tumors respond to treatment with the targeted therapy, imatinib, a selective tyrosine kinase inhibitor that inhibits specifically BCR-ABL, KIT, and PDGFRA. However, most GIST patients eventually relapse due to a secondary mutation in KIT that markedly decreases the binding affinity of imatinib. These resistance mutations invariably arise within the adenosine 5-triphosphate (ATP)-binding pocket (exons 13 and 14) or the activation loop (exons 17 and 18) of the kinase. Of the currently approved agents for GIST, none are selective targeted agents. Rather, the currently approved agents for the treatment of GIST after imatinib are multikinase inhibitors e.g., sunitinib, regorafenib, and midostaurin. In many cases, these multikinase inhibitors only weakly inhibit imatinib resistant mutants and/or the multikinase inhibitors are limited by a more complex safety profile and a small therapeutic window. A need exists for treatment of imatinib-resistant mutants of GIST.

Avapritinib (Ava, formerly BLU-285) is a potent and highly selective small molecule inhibitor of KIT and PDGFRA activation mutant loop kinases, including difficult to target KIT D816V and structurally homologous PDGFRA D842V. See, e.g., WO2015/057873, filed Oct. 15, 2014, the contents of which are hereby incorporated by reference in their entirety.

Preclinically, avapritinib has demonstrated potency across a spectrum of KIT primary and acquired resistance mutations identified in patients, including activity against KIT exon 11/17 (V560G/D816V) double mutant, as well as other KIT activation loop and JM domain mutants. ATP-binding site mutations in KIT exons 13 (V654A) and 14 (T670I) were less sensitive in vitro to avapritinib inhibition, signifying preference for wild type ATP-binding site for optimal binding of avapritinib. However, like the KIT activation loop mutants, both ATP-binding site mutants were more sensitive to avapritinib inhibition in the presence of JM domain mutants as compared to the ATP-binding site alone. Overall, avapritinib showed greater potency biochemically against all disease-relevant KIT mutants than against KIT wild type. In vivo mouse studies also indicated broad activity of avapritinib across the clinically relevant KIT mutational spectrum observed in GIST, including activity in a GIST PDX model bearing the KIT exon 11/13 double-mutant, where a dose of avapritinib at 30 mg/kg resulted in marked tumor regression. (Evans et al., A Precision Therapy Against Cancers Driven by KIT/PDGFRA Mutations Sci Transl Med. 2017 Nov. 1; 9(414)). Human pharmacokinetic data in combination with extrapolated mouse efficacy models also suggested avapritinib inhibition of a broad spectrum of primary and secondary KIT mutations at 300-400 mg once daily dosing (Heinrich et al., Abstract No. 2803523, CTOS 2017, Maui, Hi., http://www.blueprintmedicines.com/wp-content/uploads/2017/11/BLU-285-Presentation-by-Blueprint-Medicines-on-November-10-2017-at-the-CTOS-Annual-Meeting.pdf, FIG. 2). To verify avapritinib's broad spectrum of preclinical activity against KIT mutations, we conducted an analysis of exploratory biomarker samples collected during the avapritinib NAVIGATOR (NCT02508532) clinical trial, the results of which are described herein.

As the efficacy of KIT inhibitors may be strongly affected by KIT mutations that spontaneously occur in patients in response to treatment and throughout the progression of a disease, a continuing need exists for precision medicine approaches for selecting patients and identify improved methods for treating malignant diseases driven by activating KIT mutations. A precision medicine approach helps to ensure that patients receive the best treatment for their particular malignant disease and their lives are not curtailed.

### SUMMARY

The disclosure is based in part on the discovery that a patient suffering from a malignant disease, such as cancer, e.g., GIST, is responsive or nonresponsive to treatment with a KIT inhibitor, such as the selective KIT inhibitor avapritinib, when particular mutations in KIT are absent or present. In certain embodiments, a selective KIT inhibitor is capable of treating GIST when particular mutations in KIT are absent. In certain embodiments, a KIT inhibitor is not capable of treating GIST when particular mutations in KIT are present. More specifically, the inventors have discovered that the selective KIT inhibitor, avapritinib, does not provide clinical benefit in patients harboring KIT ATP binding pocket mutations (KIT V654A, N655T, and/or T670I). This is a surprising, unexpected result in view of avapritinib's broad spectrum of preclinical activity against primary and secondary KIT mutations. Thus, patients with KIT ATP binding pocket mutations should not receive avapritinib therapy and therefore be excluded by an appropriate companion diagnostic.

In one aspect, the disclosure includes methods of treating a malignant disease driven by activating mutations in KIT such as cancer, e.g., GIST, comprising: (a) obtaining a biological sample from the patient; (b) contacting the sample with a reagent that detects a KIT mutation, selected from V654A in exon 13, N655T in exon 13, and T670I in exon 14, and, if the mutation is not detected in the patient, (c) administering a KIT inhibitor. In some embodiments, the KIT inhibitor is a selective KIT inhibitor. In some embodiments, the selective KIT inhibitor is avapritinib.

In another aspect, the disclosure provides methods of predicting whether a patient suffering from a malignant disease (or whether a tumor within a patient) will be responsive to treatment with a KIT inhibitor, such as, a selective KIT inhibitor (e.g., avapritinib); the methods include: (a) obtaining a biological sample from the patient; (b) detecting the presence or absence of a KIT mutation selected from V654A in exon 13, N655T in exon 13, and T670I in exon 14 in the biological sample; and (c) if the KIT mutation is absent from the biological sample, concluding that the patient (or the tumor) will be responsive to a KIT inhibitor, and if the KIT mutation is present, concluding the patient (or the tumor) will be nonresponsive to treatment with a KIT inhibitor.

In another aspect, the disclosure provides methods of identifying a patient suffering from cancer who is likely to respond to treatment with a KIT inhibitor, such as, e.g., a selective KIT inhibitor, e.g., avapritinib, or identifying a tumor within a patient that is likely to respond to treatment with a KIT inhibitor, such as e.g., a selective KIT inhibitor, e.g., avapritinib; the methods include: (a) obtaining a biological sample from the patient; and (b) contacting the sample with a reagent that detects a KIT mutation to determine whether the KIT mutation is present in the biological sample, the KIT mutation selected from V654A, N655T, and T670I, wherein the absence of the KIT mutation indicates that the patient or tumor is likely to respond to treatment with a KIT inhibitor. In some embodiments, the KIT inhibitor is a selective KIT inhibitor, e.g., avapritinib.

In another aspect, the disclosure provides methods of identifying a patient suffering from GIST who is likely to respond to treatment with a KIT inhibitor, such as, e.g., a selective KIT inhibitor, e.g., avapritinib, or identifying a tumor within a patient that is likely to respond to treatment with a KIT inhibitor, such as a selective KIT inhibitor, e.g., avapritinib; the methods include: (a) obtaining a biological sample from the patient; and (b) contacting the sample with a reagent that detects a KIT mutation to determine whether the KIT mutation is present in the biological sample, the KIT mutation selected from V654A, N655T, and T670I, wherein the absence of the KIT mutation indicates that the patient or tumor is likely to respond to treatment with a KIT inhibitor. In some embodiments, the KIT inhibitor is a selective KIT inhibitor, e.g., avapritinib.

In another aspect, the disclosure provides methods for detecting the presence of a KIT mutation in a biological sample. The methods include the steps of: (a) obtaining a biological sample from a patient; and (b) contacting the sample with a reagent that detects the KIT mutation, the KIT mutation selected from V654A, N655T, and T670I, to determine whether the KIT mutation is present in the biological sample.

In some embodiments the biological sample can be from a patient with a malignant disease, such as a cancer patient, e.g., a GIST cancer patient, a systemic mastocytosis (SM) cancer patient e.g., advanced SM (advSM), aggressive SM (ASM), smoldering SM (SSM), SM with associated hemotologic non-mast cell lineage disease (SM-AHNMD), and mast cell leukemia (MCL), an AML (acute myeloid leukemia) cancer patient, a melanoma cancer patient, a seminoma cancer patient, a cancer patient suffering from intercranial germ cell tumors, a mediastinal B-cell lymphoma cancer patient, a patient suffering from Ewing's sarcoma, a DLBCL (diffuse large B cell lymphoma) cancer patient, dysgerminoma, MDS (myelodysplastic syndrome), NKTCL (nasal NK/T-cell lymphoma) cancer patient, a CMML (chronic myelomonocytic leukemia) cancer patient, a patient suffering from brain cancer or a patient suffering from a different cancer driven by activating mutations in KIT. In some embodiments, the biological sample can be from a patient with a malignant disease, e.g., indolent systemic mastocytosis (ISM).

In some embodiments, the KIT mutation is detected in a nucleotide encoding a KIT polypeptide or a portion thereof. In some embodiments the nucleotide is a gene, e.g., DNA. In some embodiments this nucleotide is a product of a gene (e.g., cDNA, mRNA, or variants thereof). In other embodiments, the KIT mutation is detected in a KIT polypeptide or a portion thereof. In some embodiments, the mutant KIT nucleotide encoding the V654A mutation comprises the nucleotide sequence set forth in SEQ ID NO:1 and SEQ ID NO:4 or a portion thereof (such as, e.g., the mutation site). In some embodiments, the mutant KIT polypeptide comprising the V654A mutation comprises the amino acid sequence set forth in SEQ ID NO:7 or a portion thereof (such as, e.g., the mutation site). In some embodiments, the mutant KIT nucleotide encoding the N655T mutation comprises the nucleotide sequence set forth in SEQ ID NO:2 and SEQ ID NO:5 or a portion thereof (such as, e.g., the mutation site). In some embodiments, the mutant KIT polypeptide comprising the N655T mutation comprises the amino acid sequence set forth in SEQ ID NO:8, or a portion thereof (such as, e.g., the mutation site). In some embodiments, the mutant KIT nucleotide encoding the T670I mutation comprises the nucleotide sequence set forth in SEQ ID NO:3 or SEQ ID NO:6 or a portion thereof (such as, e.g., the mutation site). In some embodiments, the mutant KIT polypeptide comprising the T670I mutation comprises the amino acid sequence set forth in SEQ ID NO:9, or a portion thereof (such as, e.g., the mutation site).

### BRIEF DESCRIPTION OF THE FIGURES

FIG. 1 is a waterfall plot that shows marked tumor reductions in GIST patients with optimal genotypes i.e., patients without V654A or T670I mutations (data cut Sep. 27, 2019).

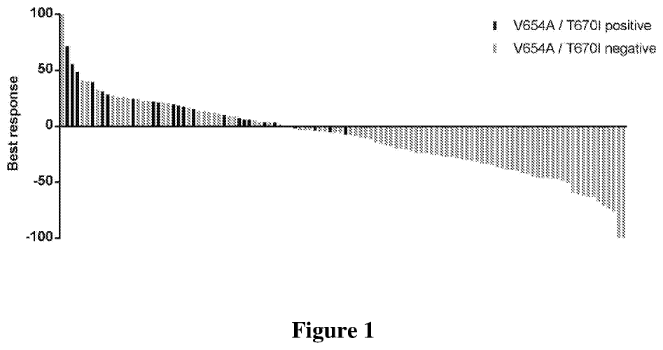

FIG. 2 is the plasma exposures required for efficacy in PDX mouse models (of various genotypes 11/17 or 11/13) layered on top of the human clinical exposures of avapritinib at 300-400 mg once daily dosing as presented at the Collective Tissue Oncology Society meeting (2017).

### DETAILED DESCRIPTION

The disclosure is based in part on the discovery of a method of treating a patient with a malignant disease by administering to the patient a KIT inhibitor. In some embodiments, the KIT inhibitor is a selective KIT inhibitor, e.g., avapritinib. The method comprises detecting the presence or absence of a KIT mutation in a biological sample obtained from the patient, and if the KIT mutation is not detected, administering treatment with a KIT inhibitor, such as a selective KIT inhibitor, e.g., avapritinib. In some embodiments the KIT mutation is V654A. In some embodiments the KIT mutation is N655T. In other embodiments the KIT mutation is T670I. In some embodiments the KIT inhibitor is the selective KIT inhibitor avapritinib. In some embodiments the patient suffers from a malignant disease characterized by the aberrant activity of KIT.

As used herein, a “malignant disease” refers to a disease in which abnormal cells divide without control and can invade nearby tissues. Malignant cells can also spread to other parts of the body through the blood or lymph system. Examples of malignant diseases are carcinoma, sarcoma, leukemia, and lymphoma. Cancer is a malignant disease. Systemic mastocytosis is a malignant disease. Indolent systemic mastocytosis is a malignant disease.

Examples of cancer include gastrointestinal stomal tumor (GIST), AML (acute myeloid leukemia), melanoma, seminoma, intercranial germ cell tumors, mediastinal B-cell lymphoma.

As used herein, an “inhibitor” refers to a compound or a pharmaceutically acceptable salt or solvate thereof that inhibits a protein e.g., an enzyme such that a reduction in activity of the protein can be observed e.g., by biochemical assay. In certain embodiments, an inhibitor has an IC50 of less than 1 mM, less than 500 nM, less than 250 nM, less than 100 nM, less than 50 nM, less than 20 nM, less than 10 nM, less than 5 nM, and less than 1 nM.

A “KIT inhibitor” refers to a compound or a pharmaceutically acceptable salt or solvate thereof that inhibits a KIT protein (wild type or mutant). Examples of KIT inhibitors include: avapritinib, DCC2618 (ripretinib), PLX9486, PLX3397, midostaurin, imatinib, sunitinib, and regorafenib. In some embodiments, a KIT inhibitor is a compound that inhibits a spectrum of KIT mutant proteins e.g., avapritinib (Evans et al. (2017)) and DCC2618 (ripretinib) (Smith et al., AACR Annual meeting, abstract 3925, poster 39, board 5). In some embodiments, a KIT inhibitor is a compound that inhibits a KIT protein produced from a KIT gene with one or more mutations in exon 11, exon 13, exon 14, and/or exon 17. In some embodiments, a KIT inhibitor is compound that inhibits a KIT mutant protein produced from a KIT gene with a mutation in exon 17. In some embodiments, a KIT inhibitor is a compound that inhibits a KIT mutant protein produced from a KIT gene with a D816 mutation. In some embodiment, a KIT inhibitor is a compound that inhibits a KIT mutant protein produced from a KIT gene with a D816V mutation. In some embodiments, a KIT inhibitor is a compound that inhibits a KIT mutant protein wherein the mutation is in the activation loop.

In some embodiments, a KIT inhibitor is a compound or pharmaceutically acceptable salt or solvate thereof that inhibits a KIT protein that has a mutation which makes the KIT protein resistant to inhibition by imatinib (“imatinib-resistant KIT protein”). Examples of a KIT inhibitor that inhibits an imatinib-resistant KIT protein include e.g., avapritinib, DCC2618 (ripretinib), and sunitinib.

As used herein, a “type I KIT inhibitor” refers to a compound or a pharmaceutically acceptable salt or solvate thereof that binds to the active confirmation of KIT protein. Examples of type I KIT inhibitors are avapritinib, midostaurin, and crenolanib.

As used herein, a “type II KIT inhibitor” refers to a compound or a pharmaceutically acceptable salt or solvate thereof that binds to the inactive conformation of KIT protein. Examples of type II KIT inhibitors that share this binding mode include imatinib, sunitinib, DCC2618 (ripretinib), and regorafenib.

As used herein, a “selective KIT inhibitor” or a “selective PDGFRA inhibitor” refers to a compound or a pharmaceutically acceptable salt or solvate thereof that selectively inhibits a KIT kinase or PDGFRA kinase over another kinase and exhibits at least a 2-fold selectivity for a KIT kinase or a PDGFRA kinase over another kinase. For example, a selective KIT inhibitor or a selective PDGFRA inhibitor exhibits at least a 10-fold selectivity; at least a 15-fold selectivity; at least a 20-fold selectivity; at least a 30-fold selectivity; at least a 40-fold selectivity; at least a 50-fold selectivity; at least a 60-fold selectivity; at least a 70-fold selectivity; at least a 80-fold selectivity; at least a 90-fold selectivity; at least 100-fold, at least 125-fold, at least 150-fold, at least 175-fold, or at least 200-fold selectivity for a KIT kinase or a PDGFRA kinase over another kinase. In some embodiments, a selective KIT inhibitor or a selective PDGFRA inhibitor exhibits at least 150-fold selectivity over another kinase, e.g., VEGFR2 (vascular endothelial growth factor receptor 2), SRC (Non-receptor protein tyrosine kinase), and FLT3 (Fms-Like Tyrosine kinase 3). See for example, Evans et al. (2017). In some embodiments, selectivity for a KIT kinase or a PDGFRA kinase over another kinase is measured in a cellular assay (e.g., a cellular assay as provided herein). In other embodiments, selectivity for a KIT kinase or a PDGFRA kinase over another kinase is measured in a biochemical assay (e.g., a biochemical assay provided in Evans, et al. (2017)). In some embodiments the selective KIT inhibitor is avapritinib. In some embodiments avapritinib is also a selective PDGFRA inhibitor. In some embodiments, a selective KIT inhibitor is a pan-KIT inhibitor.

As used herein, the suffix pan- (as in pan-KIT inhibitor) is used to indicate inhibitory activity on all isoforms of that protein. In some embodiments, the pan-KIT inhibitor is DCC2618 (ripretinib).

“Avapritinib” is (S)-1-(4-fluorophenyl)-1-(2-(4-(6-(1-methyl-1H-pyrazol-4-yl)pyrrolo[2,1-f][1,2,4]triazin-4-yl)piperazin-yl)pyrimidin-5-yl)ethan-1-amine, depicted as the following:

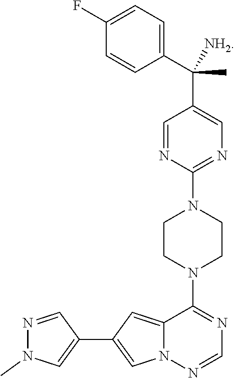 [SMILES: Cn1cc(-c2cc3c(N4CCN(c5ncc([C@@](C)(N)c6ccc(F)cc6)cn5)CC4)ncnn3c2)cn1]

“DCC2618” (also known as ripretinib) is depicted as the following:

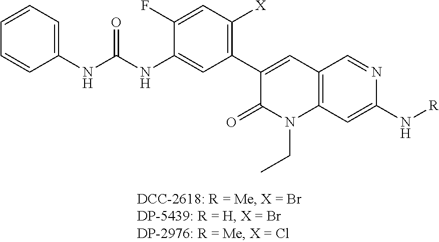

### Kit Gene Mutations and Protein Mutations

The term “KIT” refers to a human tyrosine kinase that may be referred to as mast/stem cell growth factor receptor (SCFR), proto-oncogene c-KIT, tyrosine-protein kinase Kit or CD117. As used herein, the term “KIT nucleotide” encompasses the KIT gene, KIT mRNA, KIT cDNA, and amplification products, mutations, variations, and fragments thereof. “KIT gene” is used to refer to the gene that encodes a polypeptide with KIT kinase activity, e.g., the sequence of which is located between nucleotides 55,524,085 and 55,606,881 of chromosome 4 of reference human genome hg19. “KIT transcript” refers to the transcription product of the KIT gene, one example of which has the sequence of NCBI reference sequence NM_000222.2. The term “KIT protein” refers to the polypeptide sequence that is produced by the translation of the KIT nucleotide or a portion thereof.

The term “KIT mutation”, as used herein, refers to a KIT gene, cDNA, mRNA, or protein whose sequence differs from the KIT gene sequence of human reference genome hg19, or the corresponding cDNA, mRNA, or protein. In some embodiments, when discussing KIT mutations in a nucleotide sequence that encodes a KIT polypeptide, mutations are described in terms of the change that is produced in the sequence of the polypeptide that is encoded by the nucleotide. In some embodiments the KIT mutation is V654A in exon 13. In some embodiments the genetic mutation that produces the V654A mutation is chr4:55,594,258-55,594,258 T→C (SEQ ID NO:1). See, e.g., Table #1. In some embodiments, the mutation within the mRNA transcript that produces a V654A mutation is 2048 T→C (SEQ ID NO:4). See, e.g., Table #2. In some embodiments the KIT mutation is N655T in exon 13. In some embodiments the genetic mutation that produces an N655T mutation is chr4:55,594,261-55,594,261 A→C (SEQ ID NO:2). See, e.g., Table #1. In some embodiments, the mutation within the mRNA transcript that produces an N655T mutation is 2051 A→C (SEQ ID NO:5). See, e.g., Table #2. In some embodiments the mutation is T670I in exon 14. In some embodiments, the genetic mutation that produces a T670I mutation is chr4:55,595,519-55,595,519 C→T (SEQ ID NO:3). See, e.g., Table #1. In some embodiments, the mutation within the mRNA transcript that produces a T670I mutation is 2096 C→T (SEQ ID NO:6). See, e.g., Table #2. In some embodiments, the effect of the KIT mutation on KIT kinase activity is to decrease the ability of selective KIT inhibitors, such as, e.g., avapritinib, to inhibit KIT kinase activity.

As used herein, the 5′-region is upstream of, and the 3′-region is downstream of a KIT mutation site. When a mutation occurs within a KIT gene, cDNA, or mRNA, it may be referred to as a “KIT nucleotide mutation.” When a mutation occurs within a KIT gene, it may be referred to as a “KIT gene mutation.” When a mutation occurs within a KIT amino acid sequence, it may be referred to as a “KIT protein mutation.” The term “mutant KIT” or “KIT mutation” encompasses all of the above terms. For example, a V654A “KIT mutation” encompasses a nucleotide (gene, mRNA, cDNA) encoding a polypeptide comprising the V654A mutation as well as a polypeptide comprising the V654A mutation.

TABLE 1
Sequences of Exemplary Mutant KIT Genes.

| | |
|---|---|
| SEQ ID NO: 1 | ...GTAAAGATGCTCAAGCGTAAGTTCCTGTATGGTACTGCATGCGC |
| (V654A, | |
| hg19 | TTGACATCAGTTTGCCAGTTGTGCTTTTTGCTAAAATGCATGTTTCC |
| chr4:55,594, | |
| 258-55,594,258 | AATTTTAGCGAGTGCCCATTTGACAGAACGGGAAGCCCTCATGTCTG |
| T [symbol] C) | |
| | 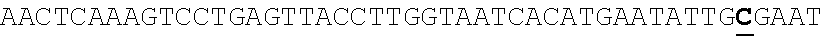 |
| | CTACTTGGAGCCTGCACCATTGGAGGTAAAGCCGTGTCCAAGCTGCC |
| | TTTTATTGTCTGTCAGGTTATCAAAACATGACATTTTAATATGATTT |
| | TGGCAATGCTAGATTATAAACTGCTTGGAAGATTTTTTTACCCAGAC |
| | TGTTGTTCTCTCTTGCTAGATTTTGTTTTCCTCATTGTTCTTAAGAA |
| | TGCAGATTTTAA... |
| SEQ ID NO: 2 | ...TAAAGATGCTCAAGCGTAAGTTCCTGTATGGTACTGCATGCGCT |
| (N655T, hg19 | |
| chr4:55,594, | TGACATCAGTTTGCCAGTTGTGCTTTTTGCTAAAATGCATGTTTCCA |
| 261-55,594,261 | |
| A [symbol] C) | ATTTTAGCGAGTGCCCATTTGACAGAACGGGAAGCCCTCATGTCTGA |
| | 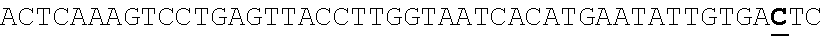 |
| | TACTTGGAGCCTGCACCATTGGAGGTAAAGCCGTGTCCAAGCTGCCT |
| | TTTATTGTCTGTCAGGTTATCAAAACATGACATTTTAATATGATTTT |
| | GGCAATGCTAGATTATAAACTGCTTGGAAGATTTTTTTACCCAGACT |
| | GTTGTTCTCTCTTGCTAGATTTTGTTTTCCTCATTGTTCTTAAGAAT |
| | GCAGATTTTAA... |
| SEQ ID NO: 3 | ...TTTTATGTTACTCCACATAAGGCTGCTTTTTTGATAAGCAGTGT |
| (T670I hg19 | |
| chr4:55,595, | TAATATATGGGATTGTATTGGGACTAAGTAGTCTGATCCACTGAAGC |
| 519-55,595,519 | |
| C [symbol] T) | TGAATATTAATGGCCATGACCACCCTTGGGTATTTTTATGGGAGGCA |
| | GAATTAATCTATATATCTCACCTTCTTTCTAACCTTTTCTTATGTGC |
| | 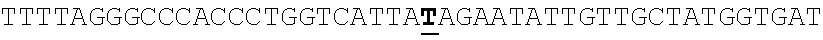 |
| | CTTTTGAATTTTTTGAGAAGAAAACGTGATTCATTTATTTGTTCAAA |
| | GCAGGAAGATCATGCAGAAGCTGCACTTTATAAGAATCTTCTGCATT |
| | CAAAGGAGTCTTCCTGGTAAGACTGATTTACATAAATAGTTAGCTGT |
| | TGACAGGCAGTT... |

TABLE 2
Sequences of Mutant KIT mRNA Transcripts

| | |
|---|---|
| SEQ ID NO: 4 | TCTGGGGGCTCGGCTTTGCCGCGCTCGCTGCACTTGGGCGAGAGCTGGAAC |
| (V654A, | |
| NM_000222.2 | GTGGACCAGAGCTCGGATCCCATCGCAGCTACCGCGATGAGAGGCGCTCGC |
| 2,048-2,048 | |
| T [symbol] C) | GGCGCCTGGGATTTTCTCTGCGTTCTGCTCCTACTGCTTCGCGTCCAGACA |
| | GGCTCTTCTCAACCATCTGTGAGTCCAGGGGAACCGTCTCCACCATCCATC |
| | CATCCAGGAAAATCAGACTTAATAGTCCGCGTGGGCGACGAGATTAGGCTG |
| | TTATGCACTGATCCGGGCTTTGTCAAATGGACTTTTGAGATCCTGGATGAA |
| | ACGAATGAGAATAAGCAGAATGAATGGATCACGGAAAAGGCAGAAGCCACC |
| | AACACCGGCAAATACACGTGCACCAACAAACACGGCTTAAGCAATTCCATT |
| | TATGTGTTTGTTAGAGATCCTGCCAAGCTTTTCCTTGTTGACCGCTCCTTG |
| | TATGGGAAAGAAGACAACGACACGCTGGTCCGCTGTCCTCTCACAGACCCA |
| | GAAGTGACCAATTATTCCCTCAAGGGGTGCCAGGGGAAGCCTCTTCCCAAG |
| | GACTTGAGGTTTATTCCTGACCCCAAGGCGGGCATCATGATCAAAAGTGTG |
| | AAACGCGCCTACCATCGGCTCTGTCTGCATTGTTCTGTGGACCAGGAGGGC |
| | AAGTCAGTGCTGTCGGAAAAATTCATCCTGAAAGTGAGGCCAGCCTTCAAA |
| | GCTGTGCCTGTTGTGTCTGTGTCCAAAGCAAGCTATCTTCTTAGGGAAGGG |
| | GAAGAATTCACAGTGACGTGCACAATAAAAGATGTGTCTAGTTCTGTGTAC |
| | TCAACGTGGAAAAGAGAAAACAGTCAGACTAAACTACAGGAGAAATATAAT |
| | AGCTGGCATCACGGTGACTTCAATTATGAACGTCAGGCAACGTTGACTATC |
| | AGTTCAGCGAGAGTTAATGATTCTGGAGTGTTCATGTGTTATGCCAATAAT |
| | ACTTTTGGATCAGCAAATGTCACAACAACCTTGGAAGTAGTAGATAAAGGA |
| | TTCATTAATATCTTCCCCATGATAAACACTACAGTATTTGTAAACGATGGA |
| | GAAAATGTAGATTTGATTGTTGAATATGAAGCATTCCCCAAACCTGAACAC |
| | CAGCAGTGGATCTATATGAACAGAACCTTCACTGATAAATGGGAAGATTAT |
| | CCCAAGTCTGAGAATGAAAGTAATATCAGATACGTAAGTGAACTTCATCTA |
| | ACGAGATTAAAAGGCACCGAAGGAGGCACTTACACATTCCTAGTGTCCAAT |
| | TCTGACGTCAATGCTGCCATAGCATTTAATGTTTATGTGAATACAAAACCA |
| | GAAATCCTGACTTACGACAGGCTCGTGAATGGCATGCTCCAATGTGTGGCA |
| | GCAGGATTCCCAGAGCCCACAATAGATTGGTATTTTTGTCCAGGAACTGAG |
| | CAGAGATGCTCTGCTTCTGTACTGCCAGTGGATGTGCAGACACTAAACTCA |
| | TCTGGGCCACCGTTTGGAAAGCTAGTGGTTCAGAGTTCTATAGATTCTAGT |
| | GCATTCAAGCACAATGGCACGGTTGAATGTAAGGCTTACAACGATGTGGGC |
| | AAGACTTCTGCCTATTTTAACTTTGCATTTAAAGGTAACAACAAAGAGCAA |
| | ATCCATCCCCACACCCTGTTCACTCCTTTGCTGATTGGTTTCGTAATCGTA |
| | GCTGGCATGATGTGCATTATTGTGATGATTCTGACCTACAAATATTTACAG |
| | AAACCCATGTATGAAGTACAGTGGAAGGTTGTTGAGGAGATAAATGGAAAC |
| | AATTATGTTTACATAGACCCAACACAACTTCCTTATGATCACAAATGGGAG |
| | TTTCCCAGAAACAGGCTGAGTTTTGGGAAAACCCTGGGTGCTGGAGCTTTC |
| | GGGAAGGTTGTTGAGGCAACTGCTTATGGCTTAATTAAGTCAGATGCGGCC |
| | ATGACTGTCGCTGTAAAGATGCTCAAGCCGAGTGCCCATTTGACAGAACGG |
| | GAAGCCCTCATGTCTGAACTCAAAGTCCTGAGTTACCTTGGTAATCACATG |
| | 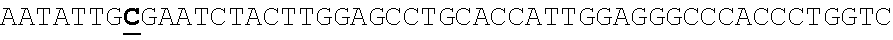 |
| | ATTACAGAATATTGTTGCTATGGTGATCTTTTGAATTTTTTGAGAAGAAAA |
| | CGTGATTCATTTATTTGTTCAAAGCAGGAAGATCATGCAGAAGCTGCACTT |
| | TATAAGAATCTTCTGCATTCAAAGGAGTCTTCCTGCAGCGATAGTACTAAT |
| | GAGTACATGGACATGAAACCTGGAGTTTCTTATGTTGTCCCAACCAAGGCC |
| | GACAAAAGGAGATCTGTGAGAATAGGCTCATACATAGAAAGAGATGTGACT |
| | CCCGCCATCATGGAGGATGACGAGTTGGCCCTAGACTTAGAAGACTTGCTG |
| | AGCTTTTCTTACCAGGTGGCAAAGGGCATGGCTTTCCTCGCCTCCAAGAAT |
| | TGTATTCACAGAGACTTGGCAGCCAGAAATATCCTCCTTACTCATGGTCGG |
| | ATCACAAAGATTTGTGATTTTGGTCTAGCCAGAGACATCAAGAATGATTCT |
| | AATTATGTGGTTAAAGGAAACGCTCGACTACCTGTGAAGTGGATGGCACCT |
| | GAAAGCATTTTCAACTGTGTATACACGTTTGAAAGTGACGTCTGGTCCTAT |
| | GGGATTTTTCTTTGGGAGCTGTTCTCTTTAGGAAGCAGCCCCTATCCTGGA |
| | ATGCCGGTCGATTCTAAGTTCTACAAGATGATCAAGGAAGGCTTCCGGATG |
| | CTCAGCCCTGAACACGCACCTGCTGAAATGTATGACATAATGAAGACTTGC |
| | TGGGATGCAGATCCCCTAAAAAGACCAACATTCAAGCAAATTGTTCAGCTA |
| | ATTGAGAAGCAGATTTCAGAGAGCACCAATCATATTTACTCCAACTTAGCA |
| | AACTGCAGCCCCAACCGACAGAAGCCCGTGGTAGACCATTCTGTGCGGATC |
| | AATTCTGTCGGCAGCACCGCTTCCTCCTCCCAGCCTCTGCTTGTGCACGAC |
| | GATGTCTGAGCAGAATCAGTGTTTGGGTCACCCCTCCAGGAATGATCTCTT |
| | CTTTTGGCTTCCATGATGGTTATTTTCTTTTCTTTCAACTTGCATCCAACT |
| | CCAGGATAGTGGGCACCCCACTGCAATCCTGTCTTTCTGAGCACACTTTAG |
| | TGGCCGATGATTTTTGTCATCAGCCACCATCCTATTGCAAAGGTTCCAACT |
| | GTATATATTCCCAATAGCAACGTAGCTTCTACCATGAACAGAAAACATTCT |
| | GATTTGGAAAAAGAGAGGGAGGTATGGACTGGGGGCCAGAGTCCTTTCCAA |
| | GGCTTCTCCAATTCTGCCCAAAAATATGGTTGATAGTTTACCTGAATAAAT |
| | GGTAGTAATCACAGTTGGCCTTCAGAACCATCCATAGTAGTATGATGATAC |
| | AAGATTAGAAGCTGAAAACCTAAGTCCTTTATGTGGAAAACAGAACATCAT |
| | TAGAACAAAGGACAGAGTATGAACACCTGGGCTTAAGAAATCTAGTATTTC |
| | ATGCTGGGAATGAGACATAGGCCATGAAAAAAATGATCCCCAAGTGTGAAC |
| | AAAAGATGCTCTTCTGTGGACCACTGCATGAGCTTTTATACTACCGACCTG |
| | GTTTTTAAATAGAGTTTGCTATTAGAGCATTGAATTGGAGAGAAGGCCTCC |
| | CTAGCCAGCACTTGTATATACGCATCTATAAATTGTCCGTGTTCATACATT |
| | TGAGGGGAAAACACCATAAGGTTTCGTTTCTGTATACAACCCTGGCATTAT |
| | GTCCACTGTGTATAGAAGTAGATTAAGAGCCATATAAGTTTGAAGGAAACA |
| | GTTAATACCATTTTTTAAGGAAACAATATAACCACAAAGCACAGTTTGAAC |
| | AAAATCTCCTCTTTTAGCTGATGAACTTATTCTGTAGATTCTGTGGAACAA |
| | GCCTATCAGCTTCAGAATGGCATTGTACTCAATGGATTTGATGCTGTTTGA |
| | CAAAGTTACTGATTCACTGCATGGCTCCCACAGGAGTGGGAAAACACTGCC |
| | ATCTTAGTTTGGATTCTTATGTAGCAGGAAATAAAGTATAGGTTTAGCCTC |
| | CTTCGCAGGCATGTCCTGGACACCGGGCCAGTATCTATATATGTGTATGTA |
| | CGTTTGTATGTGTGTAGACAAATATTTGGAGGGGTATTTTTGCCCTGAGTC |
| | CAAGAGGGTCCTTTAGTACCTGAAAAGTAACTTGGCTTTCATTATTAGTAC |
| | TGCTCTTGTTTCTTTTCACATAGCTGTCTAGAGTAGCTTACCAGAAGCTTC |
| | CATAGTGGTGCAGAGGAAGTGGAAGGCATCAGTCCCTATGTATTTGCAGTT |
| | CACCTGCACTTAAGGCACTCTGTTATTTAGACTCATCTTACTGTACCTGTT |
| | CCTTAGACCTTCCATAATGCTACTGTCTCACTGAAACATTTAAATTTTACC |
| | CTTTAGACTGTAGCCTGGATATTATTCTTGTAGTTTACCTCTTTAAAAACA |
| | AAACAAAACAAAACAAAAAACTCCCCTTCCTCACTGCCCAATATAAAAGGC |
| | AAATGTGTACATGGCAGAGTTTGTGTGTTGTCTTGAAAGATTCAGGTATGT |
| | TGCCTTTATGGTTTCCCCCTTCTACATTTCTTAGACTACATTTAGAGAACT |
| | GTGGCCGTTATCTGGAAGTAACCATTTGCACTGGAGTTCTATGCTCTCGCA |
| | CCTTTCCAAAGTTAACAGATTTTGGGGTTGTGTTGTCACCCAAGAGATTGT |
| | TGTTTGCCATACTTTGTCTGAAAAATTCCTTTGTGTTTCTATTGACTTCAA |
| | TGATAGTAAGAAAAGTGGTTGTTAGTTATAGATGTCTAGGTACTTCAGGGG |
| | CACTTCATTGAGAGTTTTGTCTTGGATATTCTTGAAAGTTTATATTTTTAT |
| | AATTTTTTCTTACATCAGATGTTTCTTTGCAGTGGCTTAATGTTTGAAATT |
| | ATTTTGTGGCTTTTTTTGTAAATATTGAAATGTAGCAATAATGTCTTTTGA |
| | ATATTCCCAAGCCCATGAGTCCTTGAAAATATTTTTTATATATACAGTAAC |
| | TTTATGTGTAAATACATAAGCGGCGTAAGTTTAAAGGATGTTGGTGTTCCA |
| | CGTGTTTTATTCCTGTATGTTGTCCAATTGTTGACAGTTCTGAAGAATTCT |
| | AATAAAATGTACATATATAAATCAAAAAAAAAAAAAAAA |
| SEQ ID NO: 5 | TCTGGGGGCTCGGCTTTGCCGCGCTCGCTGCACTTGGGCGAGAGCTGGAAC |
| (N655T, | |
| NM_000222.2 | GTGGACCAGAGCTCGGATCCCATCGCAGCTACCGCGATGAGAGGCGCTCGC |
| 2,051-2,051 | |
| A [symbol] C) | GGCGCCTGGGATTTTCTCTGCGTTCTGCTCCTACTGCTTCGCGTCCAGACA |
| | GGCTCTTCTCAACCATCTGTGAGTCCAGGGGAACCGTCTCCACCATCCATC |
| | CATCCAGGAAAATCAGACTTAATAGTCCGCGTGGGCGACGAGATTAGGCTG |
| | TTATGCACTGATCCGGGCTTTGTCAAATGGACTTTTGAGATCCTGGATGAA |
| | ACGAATGAGAATAAGCAGAATGAATGGATCACGGAAAAGGCAGAAGCCACC |
| | AACACCGGCAAATACACGTGCACCAACAAACACGGCTTAAGCAATTCCATT |
| | TATGTGTTTGTTAGAGATCCTGCCAAGCTTTTCCTTGTTGACCGCTCCTTG |
| | TATGGGAAAGAAGACAACGACACGCTGGTCCGCTGTCCTCTCACAGACCCA |
| | GAAGTGACCAATTATTCCCTCAAGGGGTGCCAGGGGAAGCCTCTTCCCAAG |
| | GACTTGAGGTTTATTCCTGACCCCAAGGCGGGCATCATGATCAAAAGTGTG |
| | AAACGCGCCTACCATCGGCTCTGTCTGCATTGTTCTGTGGACCAGGAGGGC |
| | AAGTCAGTGCTGTCGGAAAAATTCATCCTGAAAGTGAGGCCAGCCTTCAAA |
| | GCTGTGCCTGTTGTGTCTGTGTCCAAAGCAAGCTATCTTCTTAGGGAAGGG |
| | GAAGAATTCACAGTGACGTGCACAATAAAAGATGTGTCTAGTTCTGTGTAC |
| | TCAACGTGGAAAAGAGAAAACAGTCAGACTAAACTACAGGAGAAATATAAT |
| | AGCTGGCATCACGGTGACTTCAATTATGAACGTCAGGCAACGTTGACTATC |
| | AGTTCAGCGAGAGTTAATGATTCTGGAGTGTTCATGTGTTATGCCAATAAT |
| | ACTTTTGGATCAGCAAATGTCACAACAACCTTGGAAGTAGTAGATAAAGGA |
| | TTCATTAATATCTTCCCCATGATAAACACTACAGTATTTGTAAACGATGGA |
| | GAAAATGTAGATTTGATTGTTGAATATGAAGCATTCCCCAAACCTGAACAC |
| | CAGCAGTGGATCTATATGAACAGAACCTTCACTGATAAATGGGAAGATTAT |
| | CCCAAGTCTGAGAATGAAAGTAATATCAGATACGTAAGTGAACTTCATCTA |
| | ACGAGATTAAAAGGCACCGAAGGAGGCACTTACACATTCCTAGTGTCCAAT |
| | TCTGACGTCAATGCTGCCATAGCATTTAATGTTTATGTGAATACAAAACCA |
| | GAAATCCTGACTTACGACAGGCTCGTGAATGGCATGCTCCAATGTGTGGCA |
| | GCAGGATTCCCAGAGCCCACAATAGATTGGTATTTTTGTCCAGGAACTGAG |
| | CAGAGATGCTCTGCTTCTGTACTGCCAGTGGATGTGCAGACACTAAACTCA |
| | TCTGGGCCACCGTTTGGAAAGCTAGTGGTTCAGAGTTCTATAGATTCTAGT |
| | GCATTCAAGCACAATGGCACGGTTGAATGTAAGGCTTACAACGATGTGGGC |
| | AAGACTTCTGCCTATTTTAACTTTGCATTTAAAGGTAACAACAAAGAGCAA |
| | ATCCATCCCCACACCCTGTTCACTCCTTTGCTGATTGGTTTCGTAATCGTA |
| | GCTGGCATGATGTGCATTATTGTGATGATTCTGACCTACAAATATTTACAG |
| | AAACCCATGTATGAAGTACAGTGGAAGGTTGTTGAGGAGATAAATGGAAAC |
| | AATTATGTTTACATAGACCCAACACAACTTCCTTATGATCACAAATGGGAG |
| | TTTCCCAGAAACAGGCTGAGTTTTGGGAAAACCCTGGGTGCTGGAGCTTTC |
| | GGGAAGGTTGTTGAGGCAACTGCTTATGGCTTAATTAAGTCAGATGCGGCC |
| | ATGACTGTCGCTGTAAAGATGCTCAAGCCGAGTGCCCATTTGACAGAACGG |
| | GAAGCCCTCATGTCTGAACTCAAAGTCCTGAGTTACCTTGGTAATCACATG |
| | 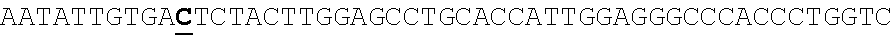 |
| | ATTACAGAATATTGTTGCTATGGTGATCTTTTGAATTTTTTGAGAAGAAAA |
| | CGTGATTCATTTATTTGTTCAAAGCAGGAAGATCATGCAGAAGCTGCACTT |
| | TATAAGAATCTTCTGCATTCAAAGGAGTCTTCCTGCAGCGATAGTACTAAT |
| | GAGTACATGGACATGAAACCTGGAGTTTCTTATGTTGTCCCAACCAAGGCC |
| | GACAAAAGGAGATCTGTGAGAATAGGCTCATACATAGAAAGAGATGTGACT |
| | CCCGCCATCATGGAGGATGACGAGTTGGCCCTAGACTTAGAAGACTTGCTG |
| | AGCTTTTCTTACCAGGTGGCAAAGGGCATGGCTTTCCTCGCCTCCAAGAAT |
| | TGTATTCACAGAGACTTGGCAGCCAGAAATATCCTCCTTACTCATGGTCGG |
| | ATCACAAAGATTTGTGATTTTGGTCTAGCCAGAGACATCAAGAATGATTCT |
| | AATTATGTGGTTAAAGGAAACGCTCGACTACCTGTGAAGTGGATGGCACCT |
| | GAAAGCATTTTCAACTGTGTATACACGTTTGAAAGTGACGTCTGGTCCTAT |
| | GGGATTTTTCTTTGGGAGCTGTTCTCTTTAGGAAGCAGCCCCTATCCTGGA |
| | ATGCCGGTCGATTCTAAGTTCTACAAGATGATCAAGGAAGGCTTCCGGATG |
| | CTCAGCCCTGAACACGCACCTGCTGAAATGTATGACATAATGAAGACTTGC |
| | TGGGATGCAGATCCCCTAAAAAGACCAACATTCAAGCAAATTGTTCAGCTA |
| | ATTGAGAAGCAGATTTCAGAGAGCACCAATCATATTTACTCCAACTTAGCA |
| | AACTGCAGCCCCAACCGACAGAAGCCCGTGGTAGACCATTCTGTGCGGATC |
| | AATTCTGTCGGCAGCACCGCTTCCTCCTCCCAGCCTCTGCTTGTGCACGAC |
| | GATGTCTGAGCAGAATCAGTGTTTGGGTCACCCCTCCAGGAATGATCTCTT |
| | CTTTTGGCTTCCATGATGGTTATTTTCTTTTCTTTCAACTTGCATCCAACT |
| | CCAGGATAGTGGGCACCCCACTGCAATCCTGTCTTTCTGAGCACACTTTAG |
| | TGGCCGATGATTTTTGTCATCAGCCACCATCCTATTGCAAAGGTTCCAACT |
| | GTATATATTCCCAATAGCAACGTAGCTTCTACCATGAACAGAAAACATTCT |
| | GATTTGGAAAAAGAGAGGGAGGTATGGACTGGGGGCCAGAGTCCTTTCCAA |
| | GGCTTCTCCAATTCTGCCCAAAAATATGGTTGATAGTTTACCTGAATAAAT |
| | GGTAGTAATCACAGTTGGCCTTCAGAACCATCCATAGTAGTATGATGATAC |
| | AAGATTAGAAGCTGAAAACCTAAGTCCTTTATGTGGAAAACAGAACATCAT |
| | TAGAACAAAGGACAGAGTATGAACACCTGGGCTTAAGAAATCTAGTATTTC |
| | ATGCTGGGAATGAGACATAGGCCATGAAAAAAATGATCCCCAAGTGTGAAC |
| | AAAAGATGCTCTTCTGTGGACCACTGCATGAGCTTTTATACTACCGACCTG |
| | GTTTTTAAATAGAGTTTGCTATTAGAGCATTGAATTGGAGAGAAGGCCTCC |
| | CTAGCCAGCACTTGTATATACGCATCTATAAATTGTCCGTGTTCATACATT |
| | TGAGGGGAAAACACCATAAGGTTTCGTTTCTGTATACAACCCTGGCATTAT |
| | GTCCACTGTGTATAGAAGTAGATTAAGAGCCATATAAGTTTGAAGGAAACA |
| | GTTAATACCATTTTTTAAGGAAACAATATAACCACAAAGCACAGTTTGAAC |
| | AAAATCTCCTCTTTTAGCTGATGAACTTATTCTGTAGATTCTGTGGAACAA |
| | GCCTATCAGCTTCAGAATGGCATTGTACTCAATGGATTTGATGCTGTTTGA |
| | CAAAGTTACTGATTCACTGCATGGCTCCCACAGGAGTGGGAAAACACTGCC |
| | ATCTTAGTTTGGATTCTTATGTAGCAGGAAATAAAGTATAGGTTTAGCCTC |
| | CTTCGCAGGCATGTCCTGGACACCGGGCCAGTATCTATATATGTGTATGTA |
| | CGTTTGTATGTGTGTAGACAAATATTTGGAGGGGTATTTTTGCCCTGAGTC |
| | CAAGAGGGTCCTTTAGTACCTGAAAAGTAACTTGGCTTTCATTATTAGTAC |
| | TGCTCTTGTTTCTTTTCACATAGCTGTCTAGAGTAGCTTACCAGAAGCTTC |
| | CATAGTGGTGCAGAGGAAGTGGAAGGCATCAGTCCCTATGTATTTGCAGTT |
| | CACCTGCACTTAAGGCACTCTGTTATTTAGACTCATCTTACTGTACCTGTT |
| | CCTTAGACCTTCCATAATGCTACTGTCTCACTGAAACATTTAAATTTTACC |
| | CTTTAGACTGTAGCCTGGATATTATTCTTGTAGTTTACCTCTTTAAAAACA |
| | AAACAAAACAAAACAAAAAACTCCCCTTCCTCACTGCCCAATATAAAAGGC |
| | AAATGTGTACATGGCAGAGTTTGTGTGTTGTCTTGAAAGATTCAGGTATGT |
| | TGCCTTTATGGTTTCCCCCTTCTACATTTCTTAGACTACATTTAGAGAACT |
| | GTGGCCGTTATCTGGAAGTAACCATTTGCACTGGAGTTCTATGCTCTCGCA |
| | CCTTTCCAAAGTTAACAGATTTTGGGGTTGTGTTGTCACCCAAGAGATTGT |
| | TGTTTGCCATACTTTGTCTGAAAAATTCCTTTGTGTTTCTATTGACTTCAA |
| | TGATAGTAAGAAAAGTGGTTGTTAGTTATAGATGTCTAGGTACTTCAGGGG |
| | CACTTCATTGAGAGTTTTGTCTTGGATATTCTTGAAAGTTTATATTTTTAT |
| | AATTTTTTCTTACATCAGATGTTTCTTTGCAGTGGCTTAATGTTTGAAATT |
| | ATTTTGTGGCTTTTTTTGTAAATATTGAAATGTAGCAATAATGTCTTTTGA |
| | ATATTCCCAAGCCCATGAGTCCTTGAAAATATTTTTTATATATACAGTAAC |
| | TTTATGTGTAAATACATAAGCGGCGTAAGTTTAAAGGATGTTGGTGTTCCA |
| | CGTGTTTTATTCCTGTATGTTGTCCAATTGTTGACAGTTCTGAAGAATTCT |
| | AATAAAATGTACATATATAAATCAAAAAAAAAAAAAAAA |
| SEQ ID NO: 6 | TCTGGGGGCTCGGCTTTGCCGCGCTCGCTGCACTTGGGCGAGAGCTGGAAC |
| (T670I, | |
| NM_000222.2 | GTGGACCAGAGCTCGGATCCCATCGCAGCTACCGCGATGAGAGGCGCTCGC |
| 2,096-2,096 | |
| C [symbol] T) | GGCGCCTGGGATTTTCTCTGCGTTCTGCTCCTACTGCTTCGCGTCCAGACA |
| | GGCTCTTCTCAACCATCTGTGAGTCCAGGGGAACCGTCTCCACCATCCATC |
| | CATCCAGGAAAATCAGACTTAATAGTCCGCGTGGGCGACGAGATTAGGCTG |
| | TTATGCACTGATCCGGGCTTTGTCAAATGGACTTTTGAGATCCTGGATGAA |
| | ACGAATGAGAATAAGCAGAATGAATGGATCACGGAAAAGGCAGAAGCCACC |
| | AACACCGGCAAATACACGTGCACCAACAAACACGGCTTAAGCAATTCCATT |
| | TATGTGTTTGTTAGAGATCCTGCCAAGCTTTTCCTTGTTGACCGCTCCTTG |
| | TATGGGAAAGAAGACAACGACACGCTGGTCCGCTGTCCTCTCACAGACCCA |
| | GAAGTGACCAATTATTCCCTCAAGGGGTGCCAGGGGAAGCCTCTTCCCAAG |
| | GACTTGAGGTTTATTCCTGACCCCAAGGCGGGCATCATGATCAAAAGTGTG |
| | AAACGCGCCTACCATCGGCTCTGTCTGCATTGTTCTGTGGACCAGGAGGGC |
| | AAGTCAGTGCTGTCGGAAAAATTCATCCTGAAAGTGAGGCCAGCCTTCAAA |
| | GCTGTGCCTGTTGTGTCTGTGTCCAAAGCAAGCTATCTTCTTAGGGAAGGG |
| | GAAGAATTCACAGTGACGTGCACAATAAAAGATGTGTCTAGTTCTGTGTAC |
| | TCAACGTGGAAAAGAGAAAACAGTCAGACTAAACTACAGGAGAAATATAAT |
| | AGCTGGCATCACGGTGACTTCAATTATGAACGTCAGGCAACGTTGACTATC |
| | AGTTCAGCGAGAGTTAATGATTCTGGAGTGTTCATGTGTTATGCCAATAAT |
| | ACTTTTGGATCAGCAAATGTCACAACAACCTTGGAAGTAGTAGATAAAGGA |
| | TTCATTAATATCTTCCCCATGATAAACACTACAGTATTTGTAAACGATGGA |
| | GAAAATGTAGATTTGATTGTTGAATATGAAGCATTCCCCAAACCTGAACAC |
| | CAGCAGTGGATCTATATGAACAGAACCTTCACTGATAAATGGGAAGATTAT |
| | CCCAAGTCTGAGAATGAAAGTAATATCAGATACGTAAGTGAACTTCATCTA |
| | ACGAGATTAAAAGGCACCGAAGGAGGCACTTACACATTCCTAGTGTCCAAT |
| | TCTGACGTCAATGCTGCCATAGCATTTAATGTTTATGTGAATACAAAACCA |
| | GAAATCCTGACTTACGACAGGCTCGTGAATGGCATGCTCCAATGTGTGGCA |
| | GCAGGATTCCCAGAGCCCACAATAGATTGGTATTTTTGTCCAGGAACTGAG |
| | CAGAGATGCTCTGCTTCTGTACTGCCAGTGGATGTGCAGACACTAAACTCA |
| | TCTGGGCCACCGTTTGGAAAGCTAGTGGTTCAGAGTTCTATAGATTCTAGT |
| | GCATTCAAGCACAATGGCACGGTTGAATGTAAGGCTTACAACGATGTGGGC |
| | AAGACTTCTGCCTATTTTAACTTTGCATTTAAAGGTAACAACAAAGAGCAA |
| | ATCCATCCCCACACCCTGTTCACTCCTTTGCTGATTGGTTTCGTAATCGTA |
| | GCTGGCATGATGTGCATTATTGTGATGATTCTGACCTACAAATATTTACAG |
| | AAACCCATGTATGAAGTACAGTGGAAGGTTGTTGAGGAGATAAATGGAAAC |
| | AATTATGTTTACATAGACCCAACACAACTTCCTTATGATCACAAATGGGAG |
| | TTTCCCAGAAACAGGCTGAGTTTTGGGAAAACCCTGGGTGCTGGAGCTTTC |
| | GGGAAGGTTGTTGAGGCAACTGCTTATGGCTTAATTAAGTCAGATGCGGCC |
| | ATGACTGTCGCTGTAAAGATGCTCAAGCCGAGTGCCCATTTGACAGAACGG |
| | GAAGCCCTCATGTCTGAACTCAAAGTCCTGAGTTACCTTGGTAATCACATG |
| | AATATTGTGAATCTACTTGGAGCCTGCACCATTGGAGGGCCCACCCTGGTC |
| | 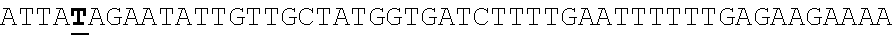 |
| | CGTGATTCATTTATTTGTTCAAAGCAGGAAGATCATGCAGAAGCTGCACTT |
| | TATAAGAATCTTCTGCATTCAAAGGAGTCTTCCTGCAGCGATAGTACTAAT |
| | GAGTACATGGACATGAAACCTGGAGTTTCTTATGTTGTCCCAACCAAGGCC |
| | GACAAAAGGAGATCTGTGAGAATAGGCTCATACATAGAAAGAGATGTGACT |
| | CCCGCCATCATGGAGGATGACGAGTTGGCCCTAGACTTAGAAGACTTGCTG |
| | AGCTTTTCTTACCAGGTGGCAAAGGGCATGGCTTTCCTCGCCTCCAAGAAT |
| | TGTATTCACAGAGACTTGGCAGCCAGAAATATCCTCCTTACTCATGGTCGG |
| | ATCACAAAGATTTGTGATTTTGGTCTAGCCAGAGACATCAAGAATGATTCT |
| | AATTATGTGGTTAAAGGAAACGCTCGACTACCTGTGAAGTGGATGGCACCT |
| | GAAAGCATTTTCAACTGTGTATACACGTTTGAAAGTGACGTCTGGTCCTAT |
| | GGGATTTTTCTTTGGGAGCTGTTCTCTTTAGGAAGCAGCCCCTATCCTGGA |
| | ATGCCGGTCGATTCTAAGTTCTACAAGATGATCAAGGAAGGCTTCCGGATG |
| | CTCAGCCCTGAACACGCACCTGCTGAAATGTATGACATAATGAAGACTTGC |
| | TGGGATGCAGATCCCCTAAAAAGACCAACATTCAAGCAAATTGTTCAGCTA |
| | ATTGAGAAGCAGATTTCAGAGAGCACCAATCATATTTACTCCAACTTAGCA |
| | AACTGCAGCCCCAACCGACAGAAGCCCGTGGTAGACCATTCTGTGCGGATC |
| | AATTCTGTCGGCAGCACCGCTTCCTCCTCCCAGCCTCTGCTTGTGCACGAC |
| | GATGTCTGAGCAGAATCAGTGTTTGGGTCACCCCTCCAGGAATGATCTCTT |
| | CTTTTGGCTTCCATGATGGTTATTTTCTTTTCTTTCAACTTGCATCCAACT |
| | CCAGGATAGTGGGCACCCCACTGCAATCCTGTCTTTCTGAGCACACTTTAG |
| | TGGCCGATGATTTTTGTCATCAGCCACCATCCTATTGCAAAGGTTCCAACT |
| | GTATATATTCCCAATAGCAACGTAGCTTCTACCATGAACAGAAAACATTCT |
| | GATTTGGAAAAAGAGAGGGAGGTATGGACTGGGGGCCAGAGTCCTTTCCAA |
| | GGCTTCTCCAATTCTGCCCAAAAATATGGTTGATAGTTTACCTGAATAAAT |
| | GGTAGTAATCACAGTTGGCCTTCAGAACCATCCATAGTAGTATGATGATAC |
| | AAGATTAGAAGCTGAAAACCTAAGTCCTTTATGTGGAAAACAGAACATCAT |
| | TAGAACAAAGGACAGAGTATGAACACCTGGGCTTAAGAAATCTAGTATTTC |
| | ATGCTGGGAATGAGACATAGGCCATGAAAAAAATGATCCCCAAGTGTGAAC |
| | AAAAGATGCTCTTCTGTGGACCACTGCATGAGCTTTTATACTACCGACCTG |
| | GTTTTTAAATAGAGTTTGCTATTAGAGCATTGAATTGGAGAGAAGGCCTCC |
| | CTAGCCAGCACTTGTATATACGCATCTATAAATTGTCCGTGTTCATACATT |
| | TGAGGGGAAAACACCATAAGGTTTCGTTTCTGTATACAACCCTGGCATTAT |
| | GTCCACTGTGTATAGAAGTAGATTAAGAGCCATATAAGTTTGAAGGAAACA |
| | GTTAATACCATTTTTTAAGGAAACAATATAACCACAAAGCACAGTTTGAAC |
| | AAAATCTCCTCTTTTAGCTGATGAACTTATTCTGTAGATTCTGTGGAACAA |
| | GCCTATCAGCTTCAGAATGGCATTGTACTCAATGGATTTGATGCTGTTTGA |
| | CAAAGTTACTGATTCACTGCATGGCTCCCACAGGAGTGGGAAAACACTGCC |
| | ATCTTAGTTTGGATTCTTATGTAGCAGGAAATAAAGTATAGGTTTAGCCTC |
| | CTTCGCAGGCATGTCCTGGACACCGGGCCAGTATCTATATATGTGTATGTA |
| | CGTTTGTATGTGTGTAGACAAATATTTGGAGGGGTATTTTTGCCCTGAGTC |
| | CAAGAGGGTCCTTTAGTACCTGAAAAGTAACTTGGCTTTCATTATTAGTAC |
| | TGCTCTTGTTTCTTTTCACATAGCTGTCTAGAGTAGCTTACCAGAAGCTTC |
| | CATAGTGGTGCAGAGGAAGTGGAAGGCATCAGTCCCTATGTATTTGCAGTT |
| | CACCTGCACTTAAGGCACTCTGTTATTTAGACTCATCTTACTGTACCTGTT |
| | CCTTAGACCTTCCATAATGCTACTGTCTCACTGAAACATTTAAATTTTACC |
| | CTTTAGACTGTAGCCTGGATATTATTCTTGTAGTTTACCTCTTTAAAAACA |
| | AAACAAAACAAAACAAAAAACTCCCCTTCCTCACTGCCCAATATAAAAGGC |
| | AAATGTGTACATGGCAGAGTTTGTGTGTTGTCTTGAAAGATTCAGGTATGT |
| | TGCCTTTATGGTTTCCCCCTTCTACATTTCTTAGACTACATTTAGAGAACT |
| | GTGGCCGTTATCTGGAAGTAACCATTTGCACTGGAGTTCTATGCTCTCGCA |
| | CCTTTCCAAAGTTAACAGATTTTGGGGTTGTGTTGTCACCCAAGAGATTGT |
| | TGTTTGCCATACTTTGTCTGAAAAATTCCTTTGTGTTTCTATTGACTTCAA |
| | TGATAGTAAGAAAAGTGGTTGTTAGTTATAGATGTCTAGGTACTTCAGGGG |
| | CACTTCATTGAGAGTTTTGTCTTGGATATTCTTGAAAGTTTATATTTTTAT |
| | AATTTTTTCTTACATCAGATGTTTCTTTGCAGTGGCTTAATGTTTGAAATT |
| | ATTTTGTGGCTTTTTTTGTAAATATTGAAATGTAGCAATAATGTCTTTTGA |
| | ATATTCCCAAGCCCATGAGTCCTTGAAAATATTTTTTATATATACAGTAAC |
| | TTTATGTGTAAATACATAAGCGGCGTAAGTTTAAAGGATGTTGGTGTTCCA |
| | CGTGTTTTATTCCTGTATGTTGTCCAATTGTTGACAGTTCTGAAGAATTCT |
| | AATAAAATGTACATATATAAATCAAAAAAAAAAAAAAAA |

TABLE 3
Amino Acid Sequnces of Mutant KIT Proteins

| | |
|---|---|
| SEQ ID | MRGARGAWDFLCVLLLLLRVQTGSSQPSVSPGEPSPPSIHPGKSDLIVRVGDEI |
| NO: 7 | |
| (V654A) | RLLCTDPGFVKWTFEILDETNENKQNEWITEKAEATNTGKYTCTNKHGLSNSIY |
| | VFVRDPAKLFLVDRSLYGKEDNDTLVRCPLTDPEVTNYSLKGCQGKPLPKDLRF |
| | IPDPKAGIMIKSVKRAYHRLCLHCSVDQEGKSVLSEKFILKVRPAFKAVPVVSV |
| | SKASYLLREGEEFTVTCTIKDVSSSVYSTWKRENSQTKLQEKYNSWHHGDFNYE |
| | RQATLTISSARVNDSGVFMCYANNTFGSANVTTTLEVVDKGFINIFPMINTTVF |
| | VNDGENVDLIVEYEAFPKPEHQQWIYMNRTFTDKWEDYPKSENESNIRYVSELH |
| | LTRLKGTEGGTYTFLVSNSDVNAAIAFNVYVNTKPEILTYDRLVNGMLQCVAAG |
| | FPEPTIDWYFCPGTEQRCSASVLPVDVQTLNSSGPPFGKLVVQSSIDSSAFKHN |
| | GTVECKAYNDVGKTSAYFNFAFKGNNKEQIHPHTLFTPLLIGFVIVAGMMCIIV |
| | MILTYKYLQKPMYEVQWKVVEEINGNNYVYIDPTQLPYDHKWEFPRNRLSFGKT |
| | LGAGAFGKVVEATAYGLIKSDAAMTVAVKMLKPSAHLTEREALMSELKVLSYLG |
| | 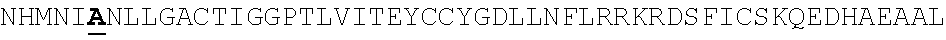 |
| | YKNLLHSKESSCSDSTNEYMDMKPGVSYVVPTKADKRRSVRIGSYIERDVTPAI |
| | MEDDELALDLEDLLSFSYQVAKGMAFLASKNCIHRDLAARNILLTHGRITKICD |
| | FGLARDIKNDSNYVVKGNARLPVKWMAPESIFNCVYTFESDVWSYGIFLWELFS |
| | LGSSPYPGMPVDSKFYKMIKEGFRMLSPEHAPAEMYDIMKTCWDADPLKRPTFK |
| | QIVQLIEKQISESTNHIYSNLANCSPNRQKPVVDHSVRINSVGSTASSSQPLLV |
| | HDDV |
| SEQ ID | MRGARGAWDFLCVLLLLLRVQTGSSQPSVSPGEPSPPSIHPGKSDLIVRVGDEI |
| NO: 8 | |
| | RLLCTDPGFVKWTFEILDETNENKQNEWITEKAEATNTGKYTCTNKHGLSNSIY |
| (N655T) | |
| | VFVRDPAKLFLVDRSLYGKEDNDTLVRCPLTDPEVTNYSLKGCQGKPLPKDLRF |
| | IPDPKAGIMIKSVKRAYHRLCLHCSVDQEGKSVLSEKFILKVRPAFKAVPVVSV |
| | SKASYLLREGEEFTVTCTIKDVSSSVYSTWKRENSQTKLQEKYNSWHHGDFNYE |
| | RQATLTISSARVNDSGVFMCYANNTFGSANVTTTLEVVDKGFINIFPMINTTVF |
| | VNDGENVDLIVEYEAFPKPEHQQWIYMNRTFTDKWEDYPKSENESNIRYVSELH |
| | LTRLKGTEGGTYTFLVSNSDVNAAIAFNVYVNTKPEILTYDRLVNGMLQCVAAG |
| | FPEPTIDWYFCPGTEQRCSASVLPVDVQTLNSSGPPFGKLVVQSSIDSSAFKHN |
| | GTVECKAYNDVGKTSAYFNFAFKGNNKEQIHPHTLFTPLLIGFVIVAGMMCIIV |
| | MILTYKYLQKPMYEVQWKVVEEINGNNYVYIDPTQLPYDHKWEFPRNRLSFGKT |
| | LGAGAFGKVVEATAYGLIKSDAAMTVAVKMLKPSAHLTEREALMSELKVLSYLG |
| | 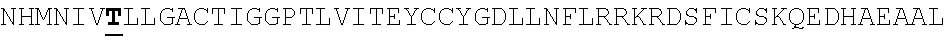 |
| | YKNLLHSKESSCSDSTNEYMDMKPGVSYVVPTKADKRRSVRIGSYIERDVTPAI |
| | MEDDELALDLEDLLSFSYQVAKGMAFLASKNCIHRDLAARNILLTHGRITKICD |
| | FGLARDIKNDSNYVVKGNARLPVKWMAPESIFNCVYTFESDVWSYGIFLWELFS |
| | LGSSPYPGMPVDSKFYKMIKEGFRMLSPEHAPAEMYDIMKTCWDADPLKRPTFK |
| | QIVQLIEKQISESTNHIYSNLANCSPNRQKPVVDHSVRINSVGSTASSSQPLLV |
| | HDDV |
| SEQ ID | MRGARGAWDFLCVLLLLLRVQTGSSQPSVSPGEPSPPSIHPGKSDLIVRVGDEI |
| NO: 9 | |
| (T670T) | RLLCTDPGFVKWTFEILDETNENKQNEWITEKAEATNTGKYTCTNKHGLSNSIY |
| | VFVRDPAKLFLVDRSLYGKEDNDTLVRCPLTDPEVTNYSLKGCQGKPLPKDLRF |
| | IPDPKAGIMIKSVKRAYHRLCLHCSVDQEGKSVLSEKFILKVRPAFKAVPVVSV |
| | SKASYLLREGEEFTVTCTIKDVSSSVYSTWKRENSQTKLQEKYNSWHHGDFNYE |
| | RQATLTISSARVNDSGVFMCYANNTFGSANVTTTLEVVDKGFINIFPMINTTVF |
| | VNDGENVDLIVEYEAFPKPEHQQWIYMNRTFTDKWEDYPKSENESNIRYVSELH |
| | LTRLKGTEGGTYTFLVSNSDVNAAIAFNVYVNTKPEILTYDRLVNGMLQCVAAG |
| | FPEPTIDWYFCPGTEQRCSASVLPVDVQTLNSSGPPFGKLVVQSSIDSSAFKHN |
| | GTVECKAYNDVGKTSAYFNFAFKGNNKEQIHPHTLFTPLLIGFVIVAGMMCIIV |
| | MILTYKYLQKPMYEVQWKVVEEINGNNYVYIDPTQLPYDHKWEFPRNRLSFGKT |
| | LGAGAFGKVVEATAYGLIKSDAAMTVAVKMLKPSAHLTEREALMSELKVLSYLG |
| | 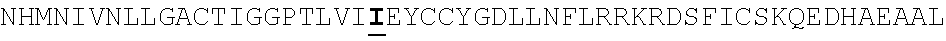 |
| | YKNLLHSKESSCSDSTNEYMDMKPGVSYVVPTKADKRRSVRIGSYIERDVTPAI |
| | MEDDELALDLEDLLSFSYQVAKGMAFLASKNCIHRDLAARNILLTHGRITKICD |
| | FGLARDIKNDSNYVVKGNARLPVKWMAPESIFNCVYTFESDVWSYGIFLWELFS |
| | LGSSPYPGMPVDSKFYKMIKEGFRMLSPEHAPAEMYDIMKTCWDADPLKRPTFK |
| | QIVQLIEKQISESTNHIYSNLANCSPNRQKPVVDHSVRINSVGSTASSSQPLLV |
| | HDDV |

As used herein, a mutant KIT nucleotide, e.g., a nucleotide comprising any one of SEQ ID NO:1-6 or a fragment or portion thereof, means that the nucleotide sequence comprises the entire mutant KIT nucleotide sequence or a fragment or portion thereof that comprises the mutation site within KIT (e.g., a nucleotide sequence that encodes a V654A, N655T, or T670I polypeptide mutation). The fragment may comprise about 1, 2, 3, 4, 5, 6, 7, 8, 9, 10, 12, 14, 16, 18, 20, 21, 22, 23, 24, 25, 26, 27, 28, 29, 30, 35, 40, 45, 50, 60, 70, 80, 90, 100, 120, 150, 175, 200, 250, 300, or more nucleotides spanning the mutation site of the KIT nucleotide. As used herein, a mutant KIT protein, e.g., comprising any one of SEQ ID NO:7-9 or a fragment or portion thereof, means an amino acid sequence that comprises the entire mutant KIT protein amino acid sequence or a fragment or portion thereof that comprises the mutation site within KIT (e.g., V654A, N655T, or T670I). The fragment may comprise about 1, 2, 3, 4, 5, 6, 7, 8, 9, 10, 12, 14, 15, 16, 18, 20, 21, 22, 23, 24, 25, 26, 27, 28, 29, 30, 35, 40, 45, 50, 75, or more amino acids spanning the mutation site.

In a particular embodiment, the disclosure provides a method for detecting the presence or absence of a mutant KIT nucleotide encoding the V654A mutation and comprising the nucleotide sequence of SEQ ID NO:1 or 4, or a fragment thereof that includes the V654A mutation site. In some embodiments, the mutant KIT nucleotide comprises a nucleotide sequence that is at least about 85%, at least about 90%, at least about 95%, at least about 97%, at least about 98%, or at least about 99% identical to SEQ ID NO:1 or 4 or a portion thereof. In a particular embodiment, the disclosure provides a mutant KIT nucleotide encoding the N655T mutation and comprising the nucleotide sequence of SEQ ID NO:2 or 5, or a fragment thereof that includes the N655T mutation site. In some embodiments, the mutant KIT nucleotide comprises a nucleotide sequence that is at least about 85%, at least about 90%, at least about 95%, at least about 97%, at least about 98%, or at least about 99% identical to SEQ ID NO:2 or 5 or a portion thereof. In a particular embodiment, the disclosure provides a mutant KIT nucleotide encoding the T670I mutation and comprising the nucleotide sequence of SEQ ID NO:3 or 6, or a fragment thereof that includes the T670I mutation site. In some embodiments, the mutant KIT nucleotide comprises a nucleotide sequence that is at least about 85%, at least about 90%, at least about 95%, at least about 97%, at least about 98%, or at least about 99% identical to SEQ ID NO:3 or 6 or a portion thereof.

The nucleic acid sequences of the mutant KIT nucleotides may be used as probes, primers, or bait to identify nucleotides from a biological sample that include, flank, or hybridize to mutant KIT genes, such as mutant KIT nucleotide V654A (for example, SEQ ID NO:1 or SEQ ID NO:4 or a portion thereof), mutant KIT nucleotide N655T (for example, SEQ ID NO:2 or SEQ ID NO:5 or a portion thereof), or mutant KIT nucleotide T670I (for example, SEQ ID NO:3 or SEQ ID NO:6 or a portion thereof), at, e.g., the mutation site. In certain embodiments, the probe, primer, or bait molecule is an oligonucleotide that allows capture, detection, and/or isolation of a mutant KIT nucleotide in a biological sample. In certain embodiments, the probes or primers derived from the nucleic acid sequences of mutant KIT nucleotides (e.g., from the mutation sites) may be used, for example, for polymerase chain reaction (PCR) amplification. The oligonucleotide can comprise a nucleotide sequence substantially complementary to a fragment of the mutant KIT nucleotide described herein. The sequence identity between the nucleic acid fragment, e.g., the oligonucleotide and the target mutant KIT nucleotide, need not be exact, so long as the sequences are sufficiently complementary to allow the capture, detection, and/or isolation of the target sequence. In one embodiment, the nucleic acid fragment is a probe or primer that includes an oligonucleotide between about 5 and 25, e.g., between 10 and 20, 10 and 15, 15 and 20, or 20 and 25, nucleotides in length that includes the mutation site of a mutant KIT nucleotide, such as, e.g., V654A (for example, SEQ ID NO:1 or SEQ ID NO:4 or a portion thereof), N655T (for example, SEQ ID NO:2 or SEQ ID NO:5 or a portion thereof), or T670I (for example, SEQ ID NO:3 or SEQ ID NO:6 or a portion thereof). In other embodiments, the nucleic acid fragment is a bait that includes an oligonucleotide between about 100 to 300 nucleotides, 130 and 230 nucleotides, or 150 and 200 nucleotides in length that includes the mutation site of a mutant KIT nucleotide, such as, e.g., V654A (for example, SEQ ID NO:1 or SEQ ID NO:4 or a portion thereof), N655T (for example, SEQ ID NO:2 or SEQ ID NO:5 or a portion thereof), or T670I (for example, SEQ ID NO:3 or SEQ ID NO:6 or a portion thereof).

In certain embodiments, the nucleic acid fragments hybridize to a nucleotide sequence that includes a mutation site, as identified with underlining and bold in Tables 1 and 2. For example, the nucleic acid fragment can hybridize to a nucleotide sequence that includes the mutation site V654A (e.g, nucleotide 70,174 of SEQ ID NO:1, corresponding to position 55,594,258 of hg19 or 2048 of SEQ ID NO:4), or the mutation site N655T (e.g., nucleotide 70,177 of SEQ ID NO:2, corresponding to position 55,594,261 of hg19 or 2051 of SEQ ID NO:5), or the mutation site T670I (e.g., nucleotide 71,435 of SEQ ID NO:3 corresponding to position 55,595,519 of hg19 or 2096 of SEQ ID NO:6), i.e., a nucleotide sequence that includes a portion of SEQ ID NO:1, 2, 3, 4, 5, or 6.

In other embodiments, the nucleic acid fragment includes a bait that comprises a nucleotide sequence that hybridizes to a mutant KIT nucleic acid molecule described herein, and thereby allows the detection, capture, and/or isolation of the nucleic acid molecule. In one embodiment, a bait is suitable for solution phase hybridization. In other embodiments, a bait includes a binding entity or detection entity, e.g., an affinity tag or fluorescent label, that allows detection, capture, and/or separation, e.g., by binding to a binding entity, of a hybrid formed by a bait and a nucleic acid hybridized to the bait.

In exemplary embodiments, the nucleic acid fragments used as bait comprise a nucleotide sequence that includes the mutation site within the mutant KIT V654A nucleotide, e.g, a nucleotide sequence within SEQ ID NO:1 comprising nucleotide 70,174 (such as, e.g., a sequence comprising nucleotides 70,173-70,175, 70,169-70,178, 70,164-70,183, 70,149-70,198, 70,124-70,223, 70,099-70,248, 70,074-70,273 of SEQ ID NO:1) or a nucleotide sequence within SEQ ID NO:4 comprising nucleotide 2048 (such as, e.g., a sequence comprising nucleotides 2047-2049, 2043-2052, 2038-2057, 2023-2072, 1998-2097, 1973-2122, or 1948-2147 of SEQ ID NO:4). In another exemplary embodiment, the nucleic acid sequences hybridize to a nucleotide sequence that includes the mutation site within the mutant KIT N655T nucleotide, e.g., a nucleotide sequence within SEQ ID NO:2 comprising nucleotide 70,177 (such as, e.g., a sequence comprising nucleotides 70,176-70,178, 70,172-70,181, 70,167-70,186, 70,152-70,201, 70,127-70,226, 70,102-70,251, or 70,077-70,276 of SEQ ID NO:2) or a nucleotide sequence within SEQ ID NO:5 comprising nucleotide 2051 (such as, e.g., a sequence comprising nucleotides 2050-2052, 2046-2055, 2041-2060, 2026-2075, 2001-2100, 1976-2125, or 1951-2150 of SEQ ID NO:5). In another exemplary embodiment, the nucleic acid sequences hybridize to a nucleotide sequence that includes the mutation site within the mutant T670I KIT nucleotide e.g., a nucleotide sequence within SEQ ID NO:3 comprising nucleotide 71,435 (such as, e.g., a sequence comprising nucleotides 71,434-71,436, 71,430-71,439, 71,425-71,444, 71,410-71,459, 71,385-71,484, 71,360-71,509, or 71,335-71,534 of SEQ ID NO:3) or a nucleotide sequence within SEQ ID NO:6 comprising nucleotide 2096 (such as, e.g., a sequence comprising nucleotides 2095-2097, 2091-2100, 2086-2105, 2071-2120, 2046-2145, 2021-2170, or 1996-2195 of SEQ ID NO:6).

Another aspect of the disclosure provides the use of mutant KIT proteins (such as, e.g., a purified or isolated KIT protein containing the V654A, N655T, or T670I mutation, biologically active or antigenic fragments thereof) for detecting and/or modulating the biological activity (such as tumorigenic activity) of a mutant KIT protein. In one embodiment, the mutant KIT protein contains a V654A mutation, and is the mutant KIT protein comprising the amino acid sequence of SEQ ID NO:7 or a fragment thereof, such as, e.g., amino acids 652-656, 649-658, 645-664, or 639-668 of SEQ ID NO:7. In another embodiment, the mutant KIT protein contains an N655T mutation and is the mutant KIT protein comprising the amino acid sequence of SEQ ID NO:8 or a fragment thereof, such as, e.g., amino acids 653-657, 650-659, 646-665, or 640-669 of SEQ ID NO:8. In another embodiment, the mutant KIT protein contains a T670I mutation, and is the mutant KIT protein comprising the amino acid sequence of SEQ ID NO:9 or a fragment thereof, such as, e.g., amino acids 668-672, 665-674, 661-680, or 655-684 of SEQ ID NO:9.

In yet another embodiment, the mutant KIT protein comprises the mutation V654A and comprises an amino acid sequence that is at least about 85%, at least about 90%, at least about 95%, at least about 97%, at least about 98%, or at least about 99% identical to SEQ ID NO:7 or a fragment thereof (e.g., amino acids 652-656, 649-658, 645-664, or 639-668 of SEQ ID NO:7). In another embodiment, the mutant KIT protein comprises the mutation N655T and comprises an amino acid sequence that is at least about 85%, at least about 90%, at least about 95%, at least about 97%, at least about 98%, or at least about 99% identical to SEQ ID NO:8 or a fragment thereof (e.g., amino acids 653-657, 650-659, 646-665, or 640-669 of SEQ ID NO:8). In yet another embodiment, the mutant KIT protein comprises the mutation T670I and comprises an amino acid sequence that is at least about 85%, at least about 90%, at least about 95%, at least about 97%, at least about 98%, or at least about 99% identical to SEQ ID NO:9 or a portion thereof (such as, e.g., amino acids 668-672, 665-674, 661-680, or 655-684 of SEQ ID NO:9).

In certain embodiments, the mutant KIT protein includes a functional kinase domain. In one exemplary embodiment, the mutant KIT protein contains a V654A mutation and includes a KIT tyrosine kinase domain or a functional fragment thereof. In another exemplary embodiment, the mutant KIT protein contains an N655T mutation and includes a KIT tyrosine kinase domain or a functional fragment thereof. In yet another embodiment, the mutant KIT protein contains a T670I mutation and includes a KIT tyrosine kinase domain or a functional fragment thereof.

In another embodiment, the mutant KIT protein is a peptide, e.g., an immunogenic peptide or protein, that contains one of the mutations as described herein. Such immunogenic peptides or proteins can be used for vaccine preparation for use in the treatment or prevention of malignant diseases caused by or exacerbated by mutant KIT nucleotides and mutant KIT proteins. In other embodiments, such immunogenic peptides or proteins can be used to raise antibodies specific to the mutant protein. In some embodiments, the mutant KIT protein is present in combination with or is further conjugated to one or more adjuvant(s) or immunogen(s), e.g., a protein capable of enhancing an immune response to the mutant KIT protein (e.g., a hapten, a toxoid, etc.). In some embodiments, the mutation within the mutant KIT protein is V654A, N655T, or T670I. In some embodiments, the mutant KIT protein comprises the mutation site of SEQ ID NO:7, 8, or 9.

Thus, another aspect of the disclosure provides an antibody that binds to a KIT protein containing a mutation (such as, e.g., V654A, N655T, or T670I) or a fragment thereof. In certain embodiments, the antibody is capable of selectively binding a mutant KIT protein (such as a mutant KIT protein containing the mutations V654A, N655T, or T670I) as compared to wild type KIT. In some embodiments, the antibody binds to an epitope comprising the mutation site of KIT (e.g., the mutation site of KIT V654A, KIT N655T, or KIT T670I). In one embodiment, the antibody binds to a KIT V654A mutant protein having the amino acid sequence of SEQ ID NO:7 or a fragment thereof, such as, e.g., amino acids 652-656, 649-658, 645-664, or 639-668 of SEQ ID NO:7. In another embodiment, the antibody binds to a KIT N655T mutant protein having the amino acid sequence of SEQ ID NO:8 or a fragment thereof, such as, e.g., amino acids 653-657, 650-659, 646-665, or 640-669 of SEQ ID NO:8. In another embodiment, the antibody binds to a KIT T670I mutant protein having the amino acid sequence of SEQ ID NO:9 or a fragment thereof, such as, e.g., amino acids 668-672, 665-674, 661-680, or 655-684 of SEQ ID NO:9.

In certain embodiments, the antibodies of the disclosure inhibit and/or neutralize the biological activity of the mutant KIT protein, and more specifically, in some embodiments, the kinase activity of the mutant KIT protein. In other embodiments, the antibodies may be used to detect a mutant KIT protein or to diagnose a patient suffering from a disease or disorder associated with the expression of a mutant KIT protein.

### Detection and Diagnostic Methods

In another aspect, the disclosure provides a method of determining the presence of a mutation, such as e.g., V654A, N655T, or T670I as described herein, within a KIT nucleotide sequence encoding a mutant KIT polypeptide, or a within a mutant KIT polypeptide. The presence of a KIT mutation in a patient suffering from a malignant disease can indicate that the disease is resistant to treatment with a KIT inhibitor. In some embodiments, the malignant disease is cancer. In some embodiments the cancer is GIST. In some embodiments, the malignant disease is mastocytosis. In some embodiments, the cancer is AML (acute myeloid leukemia). In some embodiments, the cancer is melanoma. In some embodiments, the cancer is seminoma. In some embodiments, the cancer is intercranial germ cell tumors. In some embodiments, the cancer is mediastinal B-cell lymphoma. In other embodiments, the cancer is a different cancer associated with aberrant expression or activity of a mutant KIT or overexpression of a mutant KIT.

Prior preclinical experiments have suggested that the presence of a KIT mutation, e.g., D816V exon 17, exon 13, exon 14, and exon 11 indicates a subject who may be treated with a selective KIT inhibitor. See, e.g., Evans et al. (2017). The present disclosure unexpectedly demonstrates that, contrary to those prior suggestions of broad spectrum activity of avapritinib against mutant KIT, it is the presence of the KIT mutations V654A, N655T, and/or T670I that indicates a subject suffering from a malignant disease who should not be treated with a selective KIT inhibitor, such as, e.g., avapritinib.

In one embodiment, the KIT mutation detected is in a nucleic acid or a polypeptide. The method includes detecting whether a KIT mutation is present in a nucleic acid molecule or polypeptide in a cell (e.g., a circulating cell or a cancer cell), a tissue (e.g., a tumor), or a sample (e.g., a tumor sample), from a subject. In one embodiment, the sample is a nucleic acid sample. In one embodiment, the nucleic acid sample comprises DNA, e.g., genomic DNA or cDNA, or RNA, e.g., mRNA. In other embodiments, the sample is a protein sample. The sample can be chosen from one or more of sample types: such as, e.g., tissue, e.g., cancerous tissue (e.g., a tissue biopsy), whole blood, serum, plasma, buccal scrape, fluids obtained from a Papanicolaou (Pap) test, sputum, saliva, cerebrospinal fluid, urine, stool, circulating tumor cells, circulating nucleic acids, or bone marrow. See, e.g., Hussian et al., Monitoring Daily Dynamics of Early Tumor Response to Targeted Therapy by Detecting Circulating Tumor DNA in Urine, Clin Cancer Res. 2017 Aug. 15; 23(16): 4716-4723 for an exemplary method for DNA samples from urine. See also Wang et al., Evaluation of liquid from the Papanicolaou test and other liquid biopsies for the detection of endometrial and ovarian cancers, Sci Transl Med. 2018 Mar. 21; 10(433) for an exemplary method for obtaining DNA samples from fluids obtained from Pap tests.

In some embodiments, the KIT mutation is detected in a nucleic acid molecule by one or more methods chosen from nucleic acid hybridization assays (e.g. in situ hybridization, comparative genomic hybridization, microarray, Southern blot, northern blot), amplification-based assays (e.g., PCR, PCR-RFLP assay, or real-time PCR), sequencing and genotyping (e.g. sequence-specific primers, high-performance liquid chromatography, or mass-spectrometric genotyping), and screening analysis (including metaphase cytogenetic analysis by karyotype methods).

### Hybridization Methods

In some embodiments, the reagent hybridizes to a mutant KIT nucleotide, such as, e.g., nucleotides within SEQ ID NO:1 comprising nucleotide 70,174 (such as, e.g., a sequence comprising nucleotides 70,173-70,175, 70,169-70,178, 70,164-70,183, 70,149-70,198, 70,124-70,223, 70,099-70,248, 70,074-70,273 of SEQ ID NO:1) or a nucleotide sequence within SEQ ID NO:4 comprising nucleotide 2048 (such as, e.g., a sequence comprising nucleotides 2047-2049, 2043-2052, 2038-2057, 2023-2072, 1998-2097, 1973-2122, or 1948-2147 of SEQ ID NO:4). In alternate embodiments, the reagent detects the presence of a nucleotide sequence within SEQ ID NO:2 comprising nucleotide 70,177 (such as, e.g., a sequence comprising nucleotides 70,176-70,178, 70,172-70,181, 70,167-70,186, 70,152-70,201, 70,127-70,226, 70,102-70,251, 70,077-70,276 of SEQ ID NO:2) or a nucleotide sequence within SEQ ID NO:5 comprising nucleotide 2051 (such as, e.g., a sequence comprising nucleotides 2050-2052, 2046-2055, 2041-2060, 2026-2075, 2001-2100, 1976-2125, or 1951-2150 of SEQ ID NO:5). In another exemplary embodiment, the nucleic acid sequences hybridize to a nucleotide sequence that includes the mutation site within the mutant T670I KIT nucleotide e.g., a nucleotide sequence within SEQ ID NO:3 comprising nucleotide 71,435 (such as, e.g., a sequence comprising nucleotides 71,434-71,436, 71,430-71,439, 71,425-71,444, 71,410-71,459, 71,385-71,484, 71,360-71,509, or 71,335-71,534 of SEQ ID NO:3) or a nucleotide sequence within SEQ ID NO:6 comprising nucleotide 2096 (such as, e.g., a sequence comprising nucleotides 2095-2097, 2091-2100, 2086-2105, 2071-2120, 2046-2145, 2021-2170, or 1996-2195 of SEQ ID NO:6).

In an alternate embodiment, the method includes the steps of obtaining a sample; exposing the sample to a nucleic acid probe which hybridizes to an mRNA or cDNA encoding a mutant KIT protein with the mutation V654A and that comprises amino acids 652-656, 649-658, 645-664, or 639-668 of SEQ ID NO:7. In another embodiment, the method includes the steps of obtaining a sample; exposing the sample to a nucleic acid probe which hybridizes to an mRNA or cDNA encoding a mutant KIT protein with the mutation N655T that comprises amino acids 653-657, 650-659, 646-665, or 640-669 of SEQ ID NO:8. In another embodiment, the method includes the steps of obtaining a sample; exposing the sample to a nucleic acid probe which hybridizes to an mRNA or cDNA encoding a mutant KIT protein with the mutation T670I that comprises amino acids 668-672, 665-674, 661-680, or 655-684 of SEQ ID NO:9.

Hybridization, as described throughout the specification, may be carried out under stringent conditions, e.g., medium or high stringency. See, e.g., J. Sambrook, E. F. Fritsch, and T. Maniatis, Molecular Cloning: A Laboratory Manual, Cold Spring Harbor Laboratory Pr; 2nd edition (1989); T. Brown, Hybridization Analysis of DNA Blots. Current Protocols in Molecular Biology at 21:2.10.1-2.10.16 (2001). High stringency conditions for hybridization refer to conditions under which two nucleic acids must possess a high degree of base pair homology to each other in order to hybridize. Examples of highly stringent conditions for hybridization include hybridization in 4× sodium chloride/sodium citrate (SSC), at 65 or 70° C., or hybridization in 4×SSC plus 50% formamide at about 42 or 50° C., followed by at least one, at least two, or at least three washes in 1×SSC, at 65 or 70° C. Another example of highly stringent conditions includes hybridization in 2×SSC; 10×Denhardt solution (Fikoll 400+PEG+BSA; ratio 1:1:1); 0.1% SDS; 5 mM EDTA; 50 mM Na₂HPO₄; 250 μg/ml of herring sperm DNA; 50 μg/ml of tRNA; or 0.25 M of sodium phosphate buffer, pH 7.2; 1 mM EDTA7% SDS at 60° C.; followed by washing 2×SSC, 0.1% SDS at 60° C.

The nucleic acid fragments can be detectably labeled with, e.g., a radiolabel, a fluorescent label, a bioluminescent label, a chemiluminescent label, an enzyme label, a binding pair label (e.g., biotin/streptavidin), or can include an affinity tag or identifier (e.g., an adaptor, barcode or other sequence identifier). Labeled or unlabeled nucleic acids and/or nucleic acid fragments may be used in reagents for detecting, capturing, and/or isolating mutant KIT nucleotides, such as, e.g., mutant KIT nucleotides encoding the mutation V654A (for example, SEQ ID NO:1 or SEQ ID NO:4 or a portion thereof), N655T (for example, SEQ ID NO:2 or SEQ ID NO:5 or a portion thereof), or T670I (for example, SEQ ID NO:3 or SEQ ID NO:6 or a portion thereof).

In some embodiments, the method comprises performing chromosome in situ hybridization with chromosomal DNA from a biological sample to detect the presence of a mutation within a KIT nucleotide (such as, e.g., V654A, N655T, or T670I, as disclosed herein). In some embodiments, the chromosome in situ hybridization comprises the steps of: providing a chromosome (e.g., interphase or metaphase chromosome) preparation (e.g., by attaching the chromosomes to a substrate (e.g., glass)); denaturing the chromosomal DNA (e.g., by exposure to formamide) to separate the double strands of the polynucleotides from each other; exposing the nucleic acid probe to the chromosomes under conditions to allow hybridization of the probe to the target DNA; removing unhybridized or non-specifically hybridized probes by washing; and detecting the hybridization of the probe with the target DNA. In some embodiments, the chromosome in situ hybridization is fluorescence in situ hybridization (FISH). In some embodiments, the probe is labeled directly by a fluorescent label, or indirectly by incorporation of a nucleotide containing a tag or reporter molecule (e.g., biotin, digoxigenin, or hapten) which after hybridization to the target DNA is then bound by fluorescently labeled affinity molecule (e.g., an antibody or streptavidin). In some embodiments, the hybridization of the probe with the target DNA in FISH can be visualized using a fluorescence microscope.

In other embodiments, the method comprises performing Southern blot with DNA polynucleotides from a biological sample to detect the presence of a KIT nucleotide mutation (such as, e.g., V654A, N655T, or T670I, as disclosed herein). In some embodiments, the Southern blot comprises the steps of: optionally fragmenting the polynucleotides into smaller sizes by restriction endonucleases; separating the polynucleotides by gel electrophoresis; denaturing the polynucleotides (e.g., by heat or alkali treatment) to separate the double strands of the polynucleotides from each other; transferring the polynucleotides from the gel to a membrane (e.g., a nylon or nitrocellulose membrane); immobilizing the polynucleotides to the membrane (e.g., by UV light or heat); exposing the nucleic acid probe to the polynucleotides under conditions to allow hybridization of the probe to the target DNA; removing unhybridized or non-specifically hybridized probes by washing; and detecting the hybridization of the probe with the target DNA.

### Amplification-Based Assays

In certain embodiments, the method of detecting the presence of a mutant KIT nucleotide comprises (a) performing a PCR amplification reaction with polynucleotides from a biological sample, wherein the amplification reaction utilizes a pair of primers which will amplify at least a fragment of the mutant KIT nucleotide, wherein the fragment comprises the mutation site, wherein the first primer is in sense orientation and the second primer is in antisense orientation; and (b) detecting an amplification product, wherein the presence of the amplification product is indicative of the presence of a KIT polynucleotide containing a mutation in the sample. In some embodiments, one of the primers hybridizes to a nucleotide that comprises a mutation site. In specific exemplary embodiments, the mutation in the KIT nucleotide is V654A, such as, e.g., the mutant gene of SEQ ID NO:1, or a fragment thereof, e.g., a nucleotide sequence comprising nucleotide 70,174 (such as, e.g., a sequence comprising nucleotides 70,173-70,175, 70,169-70,178, 70,164-70,183, 70,149-70,198, 70,124-70,223, 70,099-70,248, 70,074-70,273 of SEQ ID NO:1) or a nucleotide sequence within SEQ ID NO:4 comprising nucleotide 2048 (such as, e.g., a sequence comprising nucleotides 2047-2049, 2043-2052, 2038-2057, 2023-2072, 1998-2097, 1973-2122, or 1948-2147 of SEQ ID NO:4). In other exemplary embodiments, the mutation is N655T such as, e.g., the mutant nucleotide of SEQ ID NO:2 or a fragment thereof, e.g., a nucleotide sequence comprising nucleotide 70,177 (such as, e.g., a sequence comprising nucleotides 70,176-70,178, 70,172-70,181, 70,167-70,186, 70,152-70,201, 70,127-70,226, 70,102-70,251, or 70,077-70,276 of SEQ ID NO:2) or a nucleotide sequence within SEQ ID NO:5 comprising nucleotide 2051 (such as, e.g., a sequence comprising nucleotides 2050-2052, 2046-2055, 2041-2060, 2026-2075, 2001-2100, 1976-2125, or 1951-2150 of SEQ ID NO:5). In some exemplary embodiments, the KIT nucleotide encodes a mutation at T670I such as, e.g., in the mutant nucleotide SEQ ID NO:3 or a fragment thereof, e.g., a nucleotide sequence within SEQ ID NO:3 comprising nucleotide 71,435 (such as, e.g., a sequence comprising nucleotides 71,434-71,436, 71,430-71,439, 71,425-71,444, 71,410-71,459, 71,385-71,484, 71,360-71,509, or 71,335-71,534 of SEQ ID NO:3) or a nucleotide sequence within SEQ ID NO:6 comprising nucleotide 2096 (such as, e.g., a sequence comprising nucleotides 2095-2097, 2091-2100, 2086-2105, 2071-2120, 2046-2145, 2021-2170, or 1996-2195 of SEQ ID NO:6). In some embodiments, step (a) of performing a PCR amplification reaction comprises: (i) providing a reaction mixture comprising the polynucleotides (e.g., DNA or cDNA) from the biological sample, the pair of primers which will amplify at least a fragment of the mutant KIT nucleotide wherein the first primer is complementary to a sequence on the first strand of the polynucleotides and the second primer is complementary to a sequence on the second strand of the polynucleotides, a DNA polymerase, and a plurality of free nucleotides comprising adenine, thymine, cytosine, and guanine (dNTPs); (ii) heating the reaction mixture to a first predetermined temperature for a first predetermined time to separate the double strands of the polynucleotides from each other; (iii) cooling the reaction mixture to a second predetermined temperature for a second predetermined time under conditions to allow the first and second primers to hybridize with their complementary sequences on the first and second strands of the polynucleotides, and to allow the DNA polymerase to extend the primers; and (iv) repeating steps (ii) and (iii) for a predetermined number of cycles (e.g., 10, 15, 20, 25, 30, 35, 40, 45, or 50 cycles). In some embodiments, the polynucleotides from the biological sample comprise RNA, and the method further comprises performing a RT-PCR amplification reaction with the RNA to synthesize cDNA as the template for subsequent or simultaneous PCR reactions. In some embodiments, the RT-PCR amplification reaction comprises providing a reaction mixture comprising the RNA, a primer which will amplify a fragment of the RNA (e.g., a sequence-specific primer, a random primer, or oligo(dT)s), a reverse transcriptase, and dNTPs, and heating the reaction mixture to a third predetermined temperature for a third predetermined time under conditions to allow the reverse transcriptase to extend the primer.

### Sequencing and Genotyping

Another method for determining the presence of a mutation within a KIT nucleotide, which then produces a mutation in a KIT protein (such as, e.g., V645A, N655T, or T670I KIT protein mutations, as disclosed herein) includes: sequencing a portion of the nucleic acid molecule (e.g., sequencing the portion of the nucleic acid molecule that comprises the mutation site of a mutant KIT gene or gene product), thereby determining that a mutation is present in the nucleic acid molecule. In some exemplary embodiments, the KIT mutation is V654A. In other exemplary embodiments, the KIT mutation is N655T. In yet other exemplary embodiments, the KIT mutation is T670I. Optionally, the sequence acquired is compared to a reference sequence, or a wild type reference sequence, e.g., human reference genome hg19 or KIT-encoding portions thereof as described herein. In one embodiment, the sequence is determined by a next generation sequencing method. Suitable generation sequencing methods are known in the art, and non-limiting examples include technologies from Illumina, Guardant, PGDx, and Sysmex. In some embodiments the next generation sequencing method uses reversible terminator chemistry, such as, e.g., the Illumina sequencing method. See, e.g., Bentley D R et. al., Accurate whole human genome sequencing using reversible terminator chemistry. Nature. 2008 Nov. 6; 456(7218):53-9.

In some embodiments, the next generation sequencing methods employ further pre-sequencing and/or post-sequencing processing of the biological samples and/or digital sequences. See, e.g., Kim et al., Prospective blinded study of somatic mutation detection in cell-free DNA utilizing a targeted 54-gene next generation sequencing panel in metastatic solid tumor patients, Oncotarget. 2015 Nov. 24; 6(37); Lanman et al., Analytical and Clinical Validation of a Digital Sequencing Panel for Quantitative, Highly Accurate Evaluation of Cell-Free Circulating Tumor DNA, PLoS One, 2015 Oct. 16; 10(10); Phallen et al., Direct detection of early-stage cancers using circulating tumor DNA, Sci Transl Med. 2017 Aug. 16; 9(403); Kinde et al., Detection and quantification of rare mutations with massively parallel sequencing, PNAS. 2011 Jun. 11; 108(23). In some embodiments, the sequencing is automated and/or high-throughput sequencing. The method can further include acquiring, e.g., directly or indirectly acquiring, a sample, e.g., a tumor or cancer sample, from a patient.

In some embodiments, the sequencing comprises chain terminator sequencing (Sanger sequencing), comprising: providing a reaction mixture comprising a nucleic acid molecule from a biological sample, a primer complementary to a region of the template nucleic acid molecule, a DNA polymerase, a plurality of free nucleotides comprising adenine, thymine, cytosine, and guanine (dNTPs), and at least one chain terminating nucleotide (e.g., at least one di-deoxynucleotide (ddNTPs) chosen from ddATP, ddTTP, ddCTP, and ddGTP), wherein the at least one chain terminating nucleotide is present in a low concentration so that chain termination occurs randomly at any one of the positions containing the corresponding base on the DNA strand; annealing the primer to a single strand of the nucleic acid molecule; extending the primer to allow incorporation of the chain terminating nucleotide by the DNA polymerase to produce a series of DNA fragments that are terminated at positions where that particular nucleotide is used; separating the polynucleotides by electrophoresis (e.g., gel or capillary electrophoresis); and determining the nucleotide order of the template nucleic acid molecule based on the positions of chain termination on the DNA fragments. In some embodiments, the sequencing is carried out with four separate base-specific reactions, wherein the primer or the chain terminating nucleotide in each reaction is labeled with a separate fluorescent label. In other embodiments, the sequencing is carried out in a single reaction, wherein the four chain terminating nucleotides mixed in the single reaction are each labeled with a separate fluorescent label.

In some embodiments, the sequencing comprises pyrosequencing (sequencing by synthesis), comprising: (i) providing a reaction mixture comprising a nucleic acid molecule from a biological sample, a primer complementary to a region of the template nucleic acid molecule, a DNA polymerase, a first enzyme capable of converting pyrophosphate into ATP, and a second enzyme capable using ATP to generates a detectable signal (e.g., a chemiluminescent signal, such as light) in an amount that is proportional to the amount of ATP; (ii) annealing the primer to a single strand of the nucleic acid molecule; (iii) adding one of the four free nucleotides (dNTPs) to allow incorporation of the correct, complementary dNTP onto the template by the DNA polymerase and release of pyrophosphate stoichiometrically; (iv) converting the released pyrophosphate to ATP by the first enzyme; (v) generating a detectable signal by the second enzyme using the ATP; (vi) detecting the generated signal and analyzing the amount of signal generated in a pyrogram; (vii) removing the unincorporated nucleotides; and (viii) repeating steps (iii) to (vii). The method allows sequencing of a single strand of DNA, one base pair at a time, and detecting which base was actually added at each step. The solutions of each type of nucleotides are sequentially added and removed from the reaction. Light is produced only when the nucleotide solution complements the first unpaired base of the template. The order of solutions which produce detectable signals allows the determination of the sequence of the template.

In some embodiments, the method of determining the presence of a mutation in KIT (such as, e.g., V654A, N655T, or T670I, as disclosed herein) comprises analyzing a nucleic acid sample (e.g., DNA, cDNA, or RNA, or an amplification product thereof) by HPLC. The method may comprise: passing a pressurized liquid solution containing the sample through a column filled with a sorbent, wherein the nucleic acid or protein components in the sample interact differently with the sorbent, causing different flow rates for the different components; separating the components as they flow out the column at different flow rates. In some embodiments, the HPLC is chosen from, e.g., reverse-phase HPLC, size exclusion HPLC, ion-exchange HPLC, and bioaffinity HPLC.

In some embodiments, the method of determining the presence of a mutation in KIT (such as, e.g., V654A, N655T, or T670I, as disclosed herein) comprises analyzing a nucleic acid sample (e.g., DNA, cDNA, or RNA, or an amplification product thereof) by mass spectrometry. The method may comprise: ionizing the components in the sample (e.g., by chemical or electron ionization); accelerating and subjecting the ionized components to an electric or magnetic field; separating the ionized components based on their mass-to-charge ratios; and detecting the separated components by a detector capable of detecting charged particles (e.g., by an electron multiplier).

### Methods for Detecting Mutant Proteins

Another aspect of the disclosure provides a method of determining the presence of a mutation within a KIT protein (such as, e.g., V654A, N655T, or T670I, as disclosed herein) in a mammal. The method comprises the steps of obtaining a biological sample of a mammal (such as, e.g., from a human cancer), and exposing that sample to at least one reagent that detects a KIT protein containing a mutation (e.g., an antibody that recognizes the mutated KIT protein but does not recognize the wild type KIT) to determine whether a mutant KIT protein is present in the biological sample. The detection of a mutation within KIT indicates the presence of a mutated KIT protein in the mammal (such as, e.g., in the human cancer). In some embodiments, the mutant KIT protein comprises an amino acid sequence having at least 85%, 90%, 95%, 97%, 98%, or 99% identity with an amino acid sequence of any one of SEQ ID NOs 7, 8, or 9 or a fragment thereof, e.g., comprising V654A, N655T, or T670I. In some embodiments the cancer is GIST. In some embodiments, the cancer is mastocytosis. In some embodiments, the cancer is AML (acute myeloid leukemia). In some embodiments, the cancer is melanoma. In some embodiments, the cancer is seminoma. In some embodiments, the cancer is intercranial germ cell tumors. In some embodiments, the cancer is mediastinal B-cell lymphoma. In other embodiments, the cancer is a different cancer associated with aberrant expression or activity of a mutant KIT or overexpression of a mutant KIT. In some embodiments, the reagent that detects a mutant KIT protein can be detectably labeled with, e.g., a radiolabel, a fluorescent label, a bioluminescent label, a chemiluminescent label, an enzyme label, a binding pair label (e.g., biotin/streptavidin), an antigen label, or can include an affinity tag or identifier (e.g., an adaptor, barcode or other sequence identifier). In some embodiments, the labeled reagent can be detected using, e.g., autoradiography, microscopy (e.g., brightfield, fluorescence, or electron microscopy), ELISA, or immunohistochemistry. In some embodiments, the mutant KIT protein is detected in a biological sample by a method chosen from one or more of: antibody-based detection (e.g., western blot, ELISA, immunohistochemistry), size-based detection methods (e.g., HPLC or mass spectrometry), or protein sequencing.

### Antibody-Based Detection

In some embodiments, the method comprises performing a western blot with polypeptides from a biological sample to detect the presence of a mutant KIT protein (such as, e.g., V654A, N655T, or T670I, as disclosed herein). In some embodiments, the western blot comprises the steps of: separating the polypeptides by gel electrophoresis; transferring the polypeptides from the gel to a membrane (e.g., a nitrocellulose or polyvinylidene difluoride (PVDF) membrane); blocking the membrane to prevent nonspecific binding by incubating the membrane in a dilute solution of protein (e.g., 3-5% bovine serum albumin (BSA) or nonfat dry milk in Tris-Buffered Saline (TBS) or I-Block, with a minute percentage (e.g., 0.1%) of detergent, such as, e.g., Tween 20 or Triton X-100); exposing the polypeptides to at least one reagent that detects a KIT mutation (e.g., an antibody that recognizes the mutant KIT protein but does not recognize the wild type KIT protein); removing unbound or non-specifically bound reagent by washing; and detecting the binding of the reagent with the target protein. In some embodiments, the method comprises two-step detection: exposing the polypeptides to a primary antibody that specifically binds to a mutant KIT protein; removing unbound or non-specifically bound primary antibody by washing; exposing the polypeptides to a secondary antibody that recognizes the primary antibody; removing unbound or non-specifically bound secondary antibody by washing; and detecting the binding of the secondary antibody. In some embodiments, the reagent that detects a mutant KIT protein (e.g., the mutant protein specific antibody, or the secondary antibody) is directly labeled for detection. In other embodiments, the reagent is linked to an enzyme, and the method further comprises adding a substrate of the enzyme to the membrane; and developing the membrane by detecting a detectable signal produced by the reaction between the enzyme and the substrate. For example, the reagent may be linked with horseradish peroxidase to cleave a chemiluminescent agent as a substrate, producing luminescence in proportion to the amount of the target protein for detection.

In some embodiments, the method comprises performing ELISA with polypeptides from a biological sample to detect the presence of a KIT mutation (such as, e.g., V654A, N655T, or T670I, as disclosed herein). In some embodiments, the ELISA is chosen from, e.g., direct ELISA, indirect ELISA, sandwich ELISA, and competitive ELISA.

In one embodiment, the direct ELISA comprises the steps of: attaching polypeptides from a biological sample to a surface; blocking the surface to prevent nonspecific binding by incubating the surface in a dilute solution of protein; exposing the polypeptides to an antibody that specifically binds to a mutant KIT protein (e.g., an antibody that recognizes the KIT protein containing the mutation (such as, e.g., V654A, N655T, or T670I, as disclosed herein) but does not recognize the wild type KIT protein); removing unbound or non-specifically bound antibody by washing; and detecting the binding of the antibody with the target protein. In some embodiments, the antibody is directly labeled for detection. In other embodiments, the antibody is linked to an enzyme, and the method further comprises adding a substrate of the enzyme; and detecting a detectable signal produced by the reaction between the enzyme and the substrate.

In another embodiment, the indirect ELISA comprises the steps of: attaching polypeptides from a biological sample to a surface; blocking the surface to prevent nonspecific binding by incubating the surface in a dilute solution of protein; exposing the polypeptides to a primary antibody that specifically binds to a mutant KIT protein (such as, e.g., a KIT protein containing the mutation V64A, N655T, or T670I, as disclosed herein); removing unbound or non-specifically bound primary antibody by washing; exposing the polypeptides to a secondary antibody that recognizes the primary antibody; removing unbound or non-specifically bound secondary antibody by washing; and detecting the binding of the secondary antibody. In some embodiments, the secondary antibody is directly labeled for detection. In other embodiments, the secondary antibody is linked to an enzyme, and the method further comprises adding a substrate of the enzyme; and detecting a detectable signal produced by the reaction between the enzyme and the substrate.

In some embodiments, the method comprises performing immunohistochemistry with polypeptides from a biological sample to detect the presence of a KIT protein containing a mutation (such as, e.g., V654A, N655T, or T670I, as disclosed herein). In some embodiments, the immunohistochemistry comprises the steps of: fixing a cell or a tissue section (e.g., by paraformaldehyde or formalin treatment); permeabilizing the cell or tissue section to allow target accessibility; blocking the cell or tissue section to prevent nonspecific binding; exposing the cell or tissue section to at least one reagent that detects a mutant KIT protein (e.g., an antibody that recognizes the mutant KIT protein but does not recognize the wild type KIT); removing unbound or non-specifically bound reagent by washing; and detecting the binding of the reagent with the target protein. In some embodiments, the reagent is directly labeled for detection. In other embodiments, the reagent is linked to an enzyme, and the method further comprises adding a substrate of the enzyme; and detecting a detectable signal produced by the reaction between the enzyme and the substrate. In some embodiments, the immunohistochemistry may comprise the two-step detection as in the indirect ELISA.

### Size-Based Detection Methods

In some embodiments, the method of determining the presence of a mutant KIT protein (such as, e.g., a KIT protein containing a V645A, N655T, or T670I mutation, as disclosed herein) comprises analyzing a protein sample by HPLC. The method may comprise: passing a pressurized liquid solution containing the sample through a column filled with a sorbent, wherein the nucleic acid or protein components in the sample interact differently with the sorbent, causing different flow rates for the different components; separating the components as they flow out the column at different flow rates. In some embodiments, the HPLC is chosen from, e.g., reverse-phase HPLC, size exclusion HPLC, ion-exchange HPLC, and bioaffinity HPLC.

In some embodiments, the method of determining the presence of a KIT mutation (such as, e.g., V654A, N655T, or T670I, as disclosed herein) comprises analyzing a protein sample by mass spectrometry. The method may comprise: ionizing the components in the sample (e.g., by chemical or electron ionization); accelerating and subjecting the ionized components to an electric or magnetic field; separating the ionized components based on their mass-to-charge ratios; and detecting the separated components by a detector capable of detecting charged particles (e.g., by an electron multiplier).

### Methods of Treatment

Alternatively, or in combination with the detection and diagnostic methods described herein, the disclosure provides methods of identifying a patient suffering from malignant disease who is likely to respond to treatment with a KIT inhibitor, such as a selective KIT inhibitor, e.g., avapritinib, or identifying a tumor within a patient that is likely to respond to treatment with a KIT inhibitor, such as a selective KIT inhibitor, e.g., avapritinib. The methods include: (a) obtaining a biological sample from the patient; and (b) contacting the sample with a reagent that detects a KIT mutation to determine whether a KIT mutation is present in the biological sample, wherein the absence of the KIT mutation indicates that the patient or tumor is likely to respond to treatment with a KIT inhibitor such as a selective KIT inhibitor, e.g., avapritinib. In some embodiments, the presence or absence of one or more KIT mutations is detected before administering treatment. In some embodiments, the presence or absence of one of more KIT mutations is detected after administration of imatinib. In other embodiments, the presence or absence of two or more KIT mutations is detected before administering treatment. In other embodiments, the presence or absence of three or more KIT mutations is detected before administering treatment. In some embodiments the malignant disease is cancer. In certain embodiments, the cancer is GIST, mastocytosis, AML (acute myeloid leukemia), melanoma, seminoms, intercranial germ cell tumors, mediastinal B-cell lymphoma, or a different cancer associated with aberrant expression or activity of KIT.

The disclosure also includes methods of treating malignant diseases driven by activating mutations in KIT, such as cancer, e.g., GIST, the methods include: (a) obtaining a biological sample from the patient; (b) contacting the sample with a reagent that detects a KIT mutation to determine whether a KIT mutation is present in the biological sample, selected from V654A, N655T, and T670I, and, if the mutation is not detected in the patient, (c) administering a KIT inhibitor such as a selective KIT inhibitor, e.g., avapritinib, once daily in an amount of 30 mg to 400 mg.

The disclosure also includes methods of treating malignant diseases driven by activating mutations in KIT, such as cancer, e.g., GIST, the methods comprises administering an effective amount of a KIT inhibitor to the patient, and wherein the malignant disease is characterized by the absence of a KIT mutation selected from V654A in exon 13, N655T in exon 13, and T670I in exon 14.

In some embodiments, the malignant disease is characterized by the absence of one of V654A, N655T, and T670I. In some embodiments, the malignant disease is characterized by the absence of two of V654A, N655T, and T670I. In some embodiments, the malignant disease is characterized by the absence of all of V654A, N655T, and T670I.

In some embodiments, the KIT inhibitor is a selective KIT inhibitor. In one embodiment, the selective KIT inhibitor is avapritinib or ripretinib. In a specific embodiment, the KIT inhibitor is avapritinib, once daily in an amount of 30 mg to 400 mg (e.g., 300 mg). In some embodiments, the KIT inhibitor is a pan-KIT inhibitor.

In some embodiments, the activating mutation is in exon 9, 11, 17 or 18 of KIT. In a specific embodiment, the activating mutation is in exon 17 of KIT. In a more specific embodiment, the activating mutation in exon 17 of KIT is D816V, D816Y, D816F, D816K, D816H, D816A, D816G, D820A, D820E, D820G, N822K, N822H, Y823D or A829P.

The KIT inhibitor can be administered alone or in combination, with e.g., other chemotherapeutic agents or procedures, in an amount sufficient to treat a malignant disease driven by activating mutations that increase KIT expression or activity, or overexpression of KIT, by, e.g., one or more of the following: impeding growth of a cancer, causing a cancer to shrink by weight or volume, extending the expected survival time of the patient, inhibiting tumor growth, reducing tumor mass, reducing size or number of metastatic lesions, inhibiting the development of new metastatic lesions, prolonging survival, prolonging progression-free survival, prolonging time to progression, and/or enhancing quality of life.

In some embodiments, the mutant KIT nucleotide or mutant KIT protein is detected prior to, during, and/or after, a treatment of a patient with a KIT inhibitor (such as, e.g., a selective KIT inhibitor, e.g., avapritinib). In one embodiment, the mutant KIT nucleotide or mutant KIT protein is detected at the time the patient is diagnosed with a malignant disease. In other embodiments, the KIT mutation is detected at a pre-determined interval, e.g., a first point in time and at least at a subsequent point in time. In one embodiment, the KIT mutation is detected after a patient is treated with imatinib.

“PD” means progressive disease.

“SD” means stable disease.

“CR” means complete response.

“PFS” means progression free survival.

As used herein, the term “patient” refers to organisms to be treated by the methods of the present disclosure. Such organisms include, but are not limited to, mammals (e.g., murines, simians, equines, bovines, porcines, canines, felines, and the like), and in some embodiments, humans.

As used herein, the term “effective amount” refers to the amount of a KIT inhibitor, e.g., a selective KIT inhibitor, e.g., avapritinib, sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

In some embodiments, the KIT inhibitor, such as, e.g., a selective KIT inhibitor, e.g., avapritinib, is administered to a patient once daily in an amount of 30 mg to 400 mg per day. In some embodiments, the KIT inhibitor is administered to a patient once daily in an amount of 30 mg per day. In some embodiments, the KIT inhibitor is administered to a patient once daily in an amount of 40 mg per day. In some embodiments, the KIT inhibitor is administered to a patient once daily in an amount of 50 mg per day. In some embodiments, the KIT inhibitor is administered once daily to a patient in an amount of 60 mg per day. In some embodiments, the KIT inhibitor is administered to a patient once daily in an amount of 70 mg per day. In some embodiments, the KIT inhibitor is administered to a patient once daily in an amount of 80 mg per day. In some embodiments, the KIT inhibitor is administered to a patient once daily in an amount of 90 mg per day. In some embodiments, the KIT inhibitor is administered to a patient once daily in an amount of 100 mg per day. In some embodiments, the KIT inhibitor is administered to a patient once daily in an amount of 110 mg per day. In some embodiments, the KIT inhibitor is administered to a patient once daily in an amount of 120 mg per day. In some embodiments, the KIT inhibitor is administered to a patient once daily in an amount of 130 mg per day. In some embodiments, the KIT inhibitor is administered to a patient once daily in an amount of 140 mg per day. In some embodiments, the KIT inhibitor is administered to a patient once daily in an amount of 150 mg per day. In some embodiments, the KIT inhibitor is administered to a patient once daily in an amount of 160 mg per day. In some embodiments, the KIT inhibitor is administered to a patient once daily in an amount of 170 mg per day. In some embodiments, the KIT inhibitor is administered to a patient once daily in an amount of 180 mg per day. In some embodiments, the KIT inhibitor is administered to a patient once daily in an amount of 190 mg per day. In some embodiments, the KIT inhibitor is administered to a patient once daily in an amount of 200 mg per day. In some embodiments, the KIT inhibitor is administered to a patient once daily in an amount of 210 mg per day. In some embodiments, the KIT inhibitor is administered to a patient once daily in an amount of 220 mg per day. In some embodiments, the KIT inhibitor is administered to a patient once daily in an amount of 230 mg per day. In some embodiments, the KIT inhibitor is administered to a patient once daily in an amount of 240 mg per day. In some embodiments, the KIT inhibitor is administered to a patient once daily in an amount of 250 mg per day. In some embodiments, the KIT inhibitor is administered to a patient once daily in an amount of 260 mg per day. In some embodiments, the KIT inhibitor is administered to a patient once daily in an amount of 270 mg per day. In some embodiments, the KIT inhibitor is administered to a patient once daily in an amount of 280 mg per day. In some embodiments, the KIT inhibitor is administered to a patient once daily in an amount of 290 mg per day. In some embodiments, the KIT inhibitor is administered to a patient once daily in an amount of 300 mg per day. In some embodiments, the KIT inhibitor is administered to a patient once daily in an amount of 310 mg per day. In some embodiments, the KIT inhibitor is administered to a patient once daily in an amount of 320 mg per day. In some embodiments, the KIT inhibitor is administered to a patient once daily in an amount of 330 mg per day. In some embodiments, the KIT inhibitor is administered to a patient once daily in an amount of 340 mg per day. In some embodiments, the KIT inhibitor is administered to a patient once daily in an amount of 350 mg per day. In some embodiments, the selective KIT inhibitor is administered to a patient once daily in an amount of 360 mg per day. In some embodiments, the KIT inhibitor is administered to a patient once daily in an amount of 370 mg per day. In some embodiments, the KIT inhibitor is administered to a patient once daily in an amount of 380 mg per day. In some embodiments, the KIT inhibitor is administered to a patient once daily an amount of 390 mg per day. In some embodiments, the KIT inhibitor is administered to a patient once daily in an amount of 400 mg per day.

In some embodiments, a KIT inhibitor, such as e.g., a selective KIT inhibitor, avapritinib is administered to a patient once daily in an amount of 30 mg to 60 mg per day. In some embodiments, a KIT inhibitor is administered to a patient once daily in an amount of 30 mg to 90 mg per day. In some embodiments, a KIT inhibitor is administered to a patient once daily in an amount of 30 mg to 100 mg per day. In some embodiments, a KIT inhibitor is administered to a patient once daily in an amount of 30 mg to 120 mg per day. In some embodiments, a KIT inhibitor is administered to a patient once daily in an amount of 30 mg to 150 mg per day. In some embodiments, a KIT inhibitor is administered to a patient once daily in an amount of 30 mg to 200 mg per day. In some embodiments, a KIT inhibitor is administered to a patient once daily in an amount of 30 mg to 225 mg per day. In some embodiments, a KIT inhibitor is administered to a patient once daily in an amount of 30 mg to 250 mg per day. In some embodiments, a KIT inhibitor is administered to a patient once daily in an amount of 30 mg to 300 mg per day. In some embodiments the KIT inhibitor, e.g., the selective KIT inhibitor, e.g., avapritinib, is administered orally. In some embodiments, administration of the KIT inhibitor is ended due to disease progression, unacceptable toxicity or individual choice.

If the KIT inhibitor, e.g., the selective KIT inhibitor avapritinib, administered at a dose of 300 mg once daily is well-tolerated by the patient, the dose of KIT inhibitor can be increased to 400 mg once daily. If the KIT inhibitor administered at a dose of 300 mg once daily is well-tolerated by the patient for at least two consecutive treatment cycles (28 days each), for at least three consecutive treatment cycles (28 days each), or for at least four consecutive treatment cycles (28 days each), the dose of KIT inhibitor can be increased to 400 mg once daily. Avapritinib is a selective KIT inhibitor that is well-tolerated. Because avapritinib is a selective KIT inhibitor, it does not exhibit the severe dose limiting toxicities observed for other non-selective TKIs such as dermatologic, hepatic, and cardiovascular toxicities that may be a result of inhibition of a broad range of kinases.

While it is possible for the KIT inhibitor, e.g. a selective KIT inhibitor, e.g., avapritinib, to be administered alone, in some embodiments, the KIT inhibitor can be administered as a pharmaceutical formulation, wherein the KIT inhibitor is combined with one or more pharmaceutically acceptable excipients or carriers. The KIT inhibitor may be formulated for administration in any convenient way for use in human or veterinary medicine. In certain embodiments, the compound included in the pharmaceutical preparation may be active itself, or may be a prodrug, e.g., capable of being converted to an active compound in a physiological setting.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Examples of pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Solid dosage forms (e.g., capsules, tablets, pills, dragees, powders, granules and the like) can include one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents.

Liquid dosage forms can include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms for the topical or transdermal administration of avapritinib include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

When the KIT inhibitor, e.g., a selective KIT inhibitor, e.g., avapritinib, is administered as pharmaceuticals, to humans and animals, they can be given per se or as a pharmaceutical composition containing, for example, 0.1 to 99.5% (such as 0.5 to 90%) of active ingredient in combination with a pharmaceutically acceptable carrier.

The formulations can be administered topically, orally, transdermally, rectally, vaginally, parentally, intranasally, intrapulmonary, intraocularly, intravenously, intramuscularly, intraarterially, intrathecally, intracapsularly, intradermally, intraperitoneally, subcutaneously, subcuticularly, or by inhalation.

The KIT inhibitor, e.g. a selective KIT inhibitor, e.g., avapritinib, can be useful for treating malignant diseases associated with mutant KIT activity, in humans or nonhumans. For example, KIT-driven malignancies, include mastocytosis (SM), GIST (gastrointestinal stromal tumors), AML (acute myeloid leukemia), melanoma, seminoma, intercranial germ cell tumors, and mediastinal B-cell lymphoma.

Mastocytosis is subdivided into two groups of disorders: (1) cutaneous mastocytosis (CM) describes forms that are limited to the skin; and (2) systemic mastocytosis (SM) describes forms in which mast cells infiltrate extracutaneous organs, with or without skin involvement. SM is further subdivided into five forms: indolent (ISM), smoldering (SSM), aggressive (ASM), SM with associated hemotologic non-mast cell lineage disease (SM-AHNMD), and mast cell leukemia (MCL).

Diagnosis of systemic mastocytosis is based in part on histological and cytological studies of bone marrow showing infiltration by mast cells of frequently atypical morphology, which frequently abnormally express non-mast cell markers (CD25 and/or CD2). Diagnosis of SM is confirmed when bone marrow mast cell infiltration occurs in the context of one of the following: (1) abnormal mast cell morphology (spindle-shaped cells); (2) elevated level of serum tryptase above 20 ng/mL; or (3) the presence of the activating KIT D816V mutation.

Activating mutations at the D816 position are found in the vast majority of mastocytosis cases (90-98%), with the most common mutations being D816V and D816H, and D816Y. The D816V mutation is found in the activation loop of the kinase domain and leads to constitutive activation of KIT kinase.

Avapritinib can be useful for treating GIST. Complete surgical resection remains the principal treatment of choice for patients with a primary GIST. Surgery is effective in approximately 50% of patients with GIST; of the remaining patients, tumor recurrence is frequent. Primary treatment with a KIT inhibitor such as imatinib has also been shown to be sufficient for initial treatment. However, resistance to imatinib occurs within months through somatic mutation. These secondary imatinib resistant mutations are most frequently located on Exon 11, 13, 14, 17 or 18. Sunitinib is the standard of care second line treatment for most imatinib resistant tumors and is effective for those containing mutations in exons 11, 13 and 14. However, secondary KIT mutations in exons 17 and 18 are resistant to sunitinib treatment and furthermore, tumors containing tertiary resistance mutations in exon 17 and 18 emerge several months after sunitinib treatment. Regorafenib has shown promising results in a phase 3 clinical trial of imatinib, sunitinib resistant GISTs with activity against several but not all exon 17 and 18 mutations, of which D816 is one.

The KIT inhibitor, e.g. a selective KIT inhibitor, e.g., avapritinib, may also be useful in treating AML. AML patients harbor KIT mutations as well, with the majority of these mutations at the D816 position.

In addition, mutations in KIT have been linked to Ewing's sarcoma, DLBCL (diffuse large B cell lymphoma), dysgerminoma, MDS (myelodysplastic syndrome), NKTCL (nasal NK/T-cell lymphoma), CMML (chronic myelomonocytic leukemia), and brain cancers.

In some embodiments, the selective KIT inhibitor, such as, e.g., avapritinib, or a pharmaceutically acceptable salt or solvate thereof, selectively targets a KIT kinase. For example, avapritinib or a pharmaceutically acceptable salt or solvate thereof, can selectively target a KIT kinase over another kinase or non-kinase target.

In some embodiments, the KIT inhibitor, e.g., a selective KIT inhibitor, e.g., avapritinib is administered as front line therapy. In other embodiments, the KIT inhibitor is administered after a patient has been administered at least one other KIT inhibitor. In some embodiments, the KIT inhibitor is administered after the patient has been administered imatinib. In some embodiments, the KIT inhibitor is administered after the patient has been administered at least two prior therapies. In some embodiments the first prior therapy is imatinib and the second prior therapy is chosen from a tyrosine kinase inhibitor (TKI). In some embodiments, the KIT inhibitor is administered after the patient has been administered at least three prior therapies. In some embodiments, the first prior therapy is imatinib, and the second and third prior therapies are chosen from a tyrosine kinase inhibitor. In some embodiments the tyrosine kinase inhibitor is selected from sunitinib, regorafenib, sorafenib, dasatinib, and pazopanib.

As used herein, and unless otherwise specified, a “therapeutically effective amount” of a compound is an amount sufficient to provide a therapeutic benefit in the treatment or management of a malignant disease driven by activating KIT mutations that increase KIT expression or activity, or overexpression of KIT, such as, delaying or minimizing one or more symptoms associated with a malignant disease e.g., cancer or a tumor such as GIST. A therapeutically effective amount of a compound means an amount of therapeutic agent, alone or in combination with other therapeutic agents, which provides a therapeutic benefit in the treatment or management of the malignant disease. The term “therapeutically effective amount” can encompass an amount that improves overall therapy, reduces or avoids symptoms or causes of the malignant disease driven by activating KIT mutations that increase KIT expression or activity or overexpression of KIT, or enhance the therapeutic efficacy of another therapeutic agent.

All publications and patents mentioned herein are hereby incorporated by reference in their entirety as if each individual publication or patent was specifically and individually indicated to be incorporated by reference. To the extent publications and patents or patent applications incorporated by reference contradict the disclosure contained in the specification, the specification will supersede any contradictory material. Unless otherwise required by context, singular terms shall include the plural and plural terms shall include the singular. The use of “or” means “and/or” unless stated otherwise. The use of the term “including,” as well as other forms, such as “includes” and “included,” is not limiting. All ranges given in the application encompass the endpoints unless stated otherwise.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

### EXAMPLES

### Example 1

### De-Selection Markers for KIT GIST

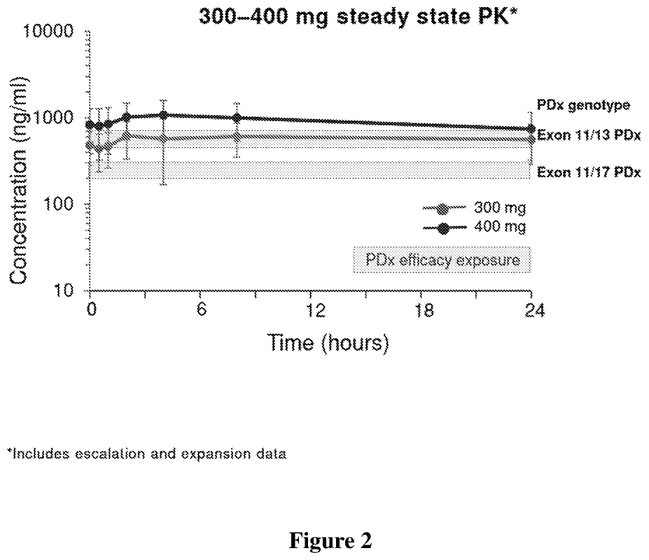

Based on preclinical biochemical data and human pharmacokinetics (PK) in combination with extrapolated mouse efficacy models, it was hypothesized that avapritinib would inhibit a broad spectrum of primary and secondary KIT mutations in patients at 300-400 mg QD dosing (FIG. 2). To verify this hypothesis, the following analysis was conducted on exploratory biomarker samples collected during the avapritinib NAVIGATOR (NCT02508532) trial (sponsored by Blueprint Medicines, Cambridge, Mass.).

The NAVIGATOR trial is an open label, nonrandomized, global, FIH Phase 1, dose-escalation/expansion study with avapritinib in advanced, unresectable GIST, which was initiated to define the safety, MTD (maximum tolerated dose), pharmacokinetics, pharmacodynamics, and preliminary antitumor activity of avapritinib. The demography and baseline patient characteristics of the NAVIGATOR clinical study are shown below.

| Parameter | All patients (n = 235) | |
|---|---|---|
| Age (years), median (range) | 61 | (25, 90) |
| GIST mutational subtype, # (n) | | |
| KIT | 72% | (170) |
| PDGFRA D842V | 24% | (56) |
| PDGFRA non-D842V | 4% | (8) |
| Metastatic disease | 97% | (228) |
| Largest target lesion size, % (n) | | |

| | | | |
|---|---|---|---|
| ≤5 | cm | 34% | (80) |
| >5-≤10 | cm | 39% | (91) |
| >10 | cm | 27% | (64) |

| No. prior unique kinase inhibitors, % (n) | PDGFRα | | KIT | |
|---|---|---|---|---|
| Median (range) | 1 | (0, 5) | 3 | (1, 7) |
| 0 | % | (11) | 0 | |
| 1 | % | (25) | 25% | (42) |
| 2 | % | (15) | 11% | (19) |
| 3 | % | (8) | 35% | (59) |
| 4 | % | (4) | 19% | (32) |
| ≥5 | % | (2) | 10% | (18) |

All statistical analyses of safety, pharmacokinetic, pharmacodynamic, and efficacy data were descriptive in nature because the primary objective of the study was to define the safety and MTD of avapritinib. The study was reviewed and approved by the institutional review board at each clinical site. Written informed consent was obtained from all patients before study entry. Key eligibility criteria for the GIST study included adult patients (≥18 years of age) with unresectable GIST who had received ≥2 kinase inhibitors, including imatinib, or patients with tumors bearing a PDGFRA D842 mutation regardless of previous therapy; Eastern Cooperative Oncology Group (ECOG) performance status of 0 to 2; and adequate bone marrow, hepatic, renal, and cardiac function. Avapritinib was administered orally, once daily, on a 4-week cycle using a 3+3 dose-escalation design. Adverse events per Common Terminology Criteria for Adverse Events (CTCAE), pharmacokinetics, ctDNA levels (Sysmex Inostics), and centrally reviewed radiographic response per RECIST 1.1 were assessed.

According to local sequencing analysis the NAVIGATOR study had enrolled 157 GIST KIT mutant patients at avapritinib doses of 300-400 mg as of the data cut on Sep. 27, 2019. Exploratory biomarker testing involved circulating tumor DNA sequencing at baseline prior to treatment with avapritinib. Although the mutation status of the tumor was known due to investigator-initiated analysis, the local sequencing results were based on archival tumor resections. The purpose of our exploratory ctDNA sequencing was to discover secondary KIT resistance mutations. Treatment induced resistance mutations occur later at disease progression and therefore are not well represented in archival tumor tissue.

To obtain a contemporaneous assessment of emerging resistance mutations, cell free DNA samples were collected predose on study day one from 112 fourth line or later patients treated with 300-400 mg avapritinib on the NAVIGATOR study, who consented to analysis of their blood. Collection involved a 20 ml blood draw followed by plasma preparation and subsequent cell free DNA extraction. Cell free DNA samples were sequenced using next gen sequencing (NGS) (Personal Genome Diagnostics (PGDx) using a customized PlasmaSELECT-60 assay). Mutations discovered in the cell free DNA were correlated with response to avapritinib as well as progression free survival. Mutations were grouped by biological mechanism to make this analysis more powerful. For example, secondary KIT mutations in the activation loop were assumed to be functionally equivalent and therefore grouped together. Similarly, secondary mutations in the ATP binding pocket of KIT, namely KIT V654A and T670I were analyzed as another group. Patients positive for any one of the mutations in these functional groups were compared to patients negative for the respective mutations. The mutation negative population also included patients with no detectable KIT mutation or any cell free DNA mutations.

| | | |
|---|---|---|
| | V654 or T670I | V654 or T670I |
| Best response | POSITIVE, | NEGATIVE, |
| n = 112 | % (n = 25) | % (n = 87) |

| | | | |
|---|---|---|---|
| ORR | 0 | 27% | (24) |

| | | |
|---|---|---|
| CR/PR | 0/0 | 1% (1)/26% (23) |

| | | | | |
|---|---|---|---|---|
| SD | 36% | (9) | 50% | (43) |
| CBR | 16% | (4) | 55% | (48) |
| PD | 64% | (16) | 23% | (20) |

Of the sequenced 4L+ patients treated at RP2D, 112 patients had at least one follow up CT scan available for RECIST response assessment. The analysis of evaluable patients revealed that no RECIST responses occurred in the 25/112 patients (22.3%) with KIT secondary mutations involving the ATP binding pocket V654A or T670I (ORR p=0.003). Moreover, median progression free survival in this ATP binding pocket mutant population was significantly shorter than in the mutation negative patients (1.8 months vs. 5.5 months, hazard ratio 7.6 CI 3.7-15.4, p<0.0001). Conversely, no significant association with either RECIST or median PFS was apparent in 41/112 (37.0%) activation loop positive patients (ORR p=0.18; PFS 5.4 vs. 3.7; hazard ratio 1.02 CI 0.67-1.56, p<0.9).

We determined that the initial efficacious dose prediction required adjustment. To the effect that even at doses of 300-400 mg QD, avapritinib does not provide clinical benefit in patients harboring KIT ATP binding pocket mutations (KIT V654A and T670I). Thus, in unexpected contrast to previous reports, patients with KIT ATP binding pocket mutations should not receive avapritinib therapy and therefore be excluded by an appropriate companion diagnostic.

1. A method for treating a patient suffering from a malignant disease driven by activating mutations in KIT, said method comprising:
(a) obtaining a biological sample from the patient;
(b) detecting the presence or absence of a KIT mutation selected from V654A in exon 13, N655T in exon 13, and T670I in exon 14 in the biological sample; and
(c) administering a KIT inhibitor to the patient if the mutation is not detected.

2. The method of claim 1, wherein the KIT inhibitor is administered if one of V654A, N655T, and T670I is not detected.

3. The method of claim 1, wherein the KIT inhibitor is administered if two of V654A, N655T, and T670I are not detected.

4. The method of claim 1, wherein the KIT inhibitor is administered if none of V654A, N655T, and T670I are detected.

5-15. (canceled)

16. The method of claim 1, wherein the malignant disease is cancer.

17. The method of claim 16, wherein the cancer is gastrointestinal stromal tumor (GIST).

18. The method of claim 16, wherein the cancer is selected from AML (acute myeloid leukemia), melanoma, seminoma, intercranial germ cell tumors, mediastinal B-cell lymphoma, Ewing's sarcoma, DLBCL (diffuse large B cell lymphoma), dysgerminoma, MDS (myelodysplastic syndrome), NKTCL (nasal NK/T-cell lymphoma), CMML (chronic myelomonocytic leukemia), brain cancers and systemic mastocytosis (smoldering (SSM), aggressive (ASM), SM with associated hemotologic non-mast cell lineage disease (SM-AHNMD), and mast cell leukemia (MCL).

19-23. (canceled)

24. The method of claim 1, wherein the KIT inhibitor is avapritinib.

25-30. (canceled)

31. A method of predicting whether a patient suffering from a malignant disease will be responsive to treatment with a KIT inhibitor, comprising:
(a) obtaining a biological sample from a patient;
(b) detecting the presence or absence of a KIT mutation selected from V654A in exon 13, N655T in exon 13, and T670I in exon 14 in the biological sample; and
(c) if the KIT mutation is absent from the biological sample, concluding that the patient will be responsive to a KIT inhibitor, and if the KIT mutation is present, concluding the patient will be nonresponsive to treatment with a KIT inhibitor.

32. The method of claim 31, wherein if one of V654A, N655T, and T670I is not detected, concluding that the patient will be responsive to treatment with a KIT inhibitor.

33. The method of claim 31, wherein if two of V654A, N655T, and T670I are not detected, concluding that the patient will be responsive to treatment with a KIT inhibitor.

34. The method of claim 31, wherein if none of V654A, N655T, and T670I are detected, concluding that the patient will be responsive to treatment with a KIT inhibitor.

35-43. (canceled)

44. The method of claim 31, wherein the malignant disease is cancer.

45. The method of claim 44, wherein the cancer is gastrointestinal stromal tumor (GIST).

46. The method of claim 44, wherein the cancer is selected from AML (acute myeloid leukemia), melanoma, seminoma, intercranial germ cell tumors, mediastinal B-cell lymphoma, Ewing's sarcoma, DLBCL (diffuse large B cell lymphoma), dysgerminoma, MDS (myelodysplastic syndrome), NKTCL (nasal NK/T-cell lymphoma), CMML (chronic myelomonocytic leukemia), brain cancers and systemic mastocytosis (smoldering (SSM), aggressive (ASM), SM with associated hemotologic non-mast cell lineage disease (SM-AHNMD), and mast cell leukemia (MCL).

47-53. (canceled)

54. A method of predicting whether a tumor will be responsive to treatment with a KIT inhibitor, comprising:
(a) obtaining a biological sample from a patient suffering from a cancer;
(b) detecting the presence or absence of a KIT mutation selected from V654A in exon 13, N655T in exon 13, and T670I in exon 14 in the biological sample; and
(c) if the KIT mutation is absent from the biological sample, concluding that the tumor will be responsive to treatment with a KIT inhibitor, and if the KIT mutation is present, concluding the tumor will be nonresponsive to treatment with a KIT inhibitor.

55. The method of claim 54, wherein if one of V654A, N655T, and T670I is not detected, concluding that the tumor will be responsive to treatment with a KIT inhibitor.

56. The method of claim 54, wherein if two of V654A, N655T, and T670I are not detected, concluding that the tumor will be responsive to treatment with a KIT inhibitor.

57. The method of claim 54, wherein if none of V654A, N655T, and T670I are detected, concluding that the tumor will be responsive to treatment with a KIT inhibitor.

58-65. (canceled)

66. The method of claim 65, wherein the cancer is gastrointestinal stromal tumor (GIST).

67-97. (canceled)
